(19) United States
(12) Patent Application Publication
(10) Pub. No.: US 2012/0053547 A1
(43) Pub. Date: Mar. 1, 2012

(54) Absorbent Composite With A Resilient Coform Layer

(76) Inventors: Karyn Clare Schroeder, Neenah, WI (US); Garry Roland Woltman, Appleton, WI (US); David Martin Jackson, Alpharetta, GA (US); David Arthur Fell, Neenah, WI (US); Kathryn Lynn Veith, Fremont, WI (US); Tammy Joy Nettekoven, Neenah, WI (US)

(21) Appl. No.: 12/872,190

(22) Filed: Aug. 31, 2010

(51) Int. Cl. A61F 13/53; B32B 37/14

(52) U.S. Cl. 604/369; 604/367; 604/372; 156/244.11

(57) ABSTRACT

An absorbent composite disposed in an absorbent article between a topsheet and a backsheet is presented, the absorbent composite including a first intake layer disposed between the topsheet and the backsheet, and a retention layer disposed between the topsheet and the backsheet, wherein one of the first intake layer and the retention layer includes a resilient coform material. When the first intake layer includes a resilient coform material, the retention layer includes one of a high-density, hydrogen-bonded, fluff/superabsorbent polymer material, a spunlace material, a superabsorbent polymer/adhesive composite material, and a foam material. The absorbent composite can further include a distribution layer disposed between the topsheet and the backsheet, the distribution layer including one of a meltblown microfiber material, a spunlace material, and a foam material.

### BACKGROUND

The development of highly absorbent articles for urine, blood, and blood-based fluids such as incontinence pads and garments, catamenial pads (e.g., sanitary napkins), tampons, wound dressings, bandages, and surgical drapes can be challenging. In the case of incontinence and catamenial pads, for example, consumers have come to expect a high level of performance in terms of comfort and fit, retention of fluid, and minimal staining. Above all, leakage of fluid from the pad onto undergarments is regarded as unacceptable. Improving the performance of such pads continues to be a formidable undertaking, although a number of improvements have been made in both structures and materials used in such structures. Eliminating leakage, particularly along the inside of the thighs, without compromising fit and comfort, has not always met the desired needs of the consumer.

The absorbent structures of current pads have typically comprised one or more fibrous layers for acquiring the discharged fluid from the permeable topsheet and distributing it to an underlying storage area. Absorbent structures for relatively thin versions of prior products usually include a fluid acquisition or intake layer that is adjacent to the permeable topsheet. This intake layer typically is made from an air-laid web or a synthetic nonwoven. Underlying this intake layer is the main absorbent core that is typically made from an air-laid or wet-laid web.

Prior absorbent structures made from fibrous layers have a number of problems. One is the difficulty in ensuring adequate topsheet dryness. Such structures have also had a greater chance of causing clothing and body soiling. This is because the absorbent structure lacks resilience, leading to bunching of the pad. This lack of resilience, and consequent bunching, has also caused these prior pads to provide poorer fit and comfort for the user. The issue that conventional absorbent structures and conventional absorbent fibrous webs have not solved this problem was recognized in U.S. Pat. No. 5,849,805 to Dyer.

One attempted solution replaced fibrous intake and absorbent layers with foam, such as the INFINICEL foam used in ALWAYS INFINITY Regular pads available from The Procter and Gamble Company of Cincinnati, Ohio. Such foams tend to be more expensive than fibrous webs.

Coform nonwoven webs, which are composites of a matrix of meltblown fibers and an absorbent material (e.g., fluff pulp fibers), have been used as an absorbent layer in a wide variety of applications, including absorbent articles, absorbent dry wipes, wet wipes, and mops. Most conventional coform webs employ meltblown fibers formed from polypropylene homopolymers. One problem sometimes experienced with such coform materials, however, is that coform materials might not be sufficiently resilient when subjected to bending forces. For example, when a coform wiper is crumpled, the coform material might not return to its original flat, unwrinkled state. As another example, a coform material used as an absorbent core in personal care absorbent product can have a tendency for bunching.

As such, a need currently exists for an improved coform nonwoven web for use in a variety of applications that shows improved resistance to bending forces and demonstrates a tendency to return to a flat state after being folded. Such an improved coform nonwoven web can be combined with various other materials to produce a next-generation absorbent composite for use in personal care absorbent articles.

### SUMMARY

The present inventors undertook intensive research and development efforts with respect to improving absorbent articles and have developed absorbent composites for use in an absorbent core that has adequate wet and dry resilience and adequate absorbency, without the primary use of expensive foams. The present inventors also found that they can tailor these properties through combining resilient coform with other materials to deliver enhanced resiliency and absorbency properties.

The present disclosure provides an absorbent composite disposed in an absorbent article between a topsheet and a backsheet, the absorbent composite including a first intake layer disposed between the topsheet and the backsheet, and a retention layer disposed between the topsheet and the backsheet, wherein one of the first intake layer and the retention layer includes a resilient coform material. When the first intake layer includes a resilient coform material, the retention layer includes one of a high-density, hydrogen-bonded, fluff/superabsorbent polymer material, a spunlace material, a superabsorbent polymer/adhesive composite material, and a foam material. The absorbent composite can further include a distribution layer disposed between the topsheet and the backsheet, the distribution layer including one of a meltblown microfiber material, a spunlace material, and a foam material.

The present disclosure also provides an absorbent composite disposed in an absorbent article between a topsheet and a backsheet, the absorbent composite including a first intake layer including one of a coform material, a resilient coform material, an airlaid material, a bonded-carded web (BCW) material, and a foam material, and a retention layer disposed between the topsheet and the backsheet, the retention layer including one of a coform material, a resilient coform material, an airlaid material, a high-density, hydrogen-bonded, fluff/superabsorbent polymer material, a spunlace material, a superabsorbent polymer/adhesive composite material, and a foam material, wherein one of the first intake layer and the retention layer includes a resilient coform material.

The present disclosure also provides an absorbent composite disposed in an absorbent article between a topsheet and a backsheet, the absorbent composite including a first intake layer including a resilient coform material, and a retention layer disposed between the topsheet and the backsheet, the retention layer including one of a coform material, a resilient coform material, an airlaid material, a high-density, hydrogen-bonded, fluff/superabsorbent polymer material, a spunlace material, a superabsorbent polymer/adhesive composite material, and a foam material.

The present disclosure also provides an absorbent personal care article having a topsheet and a backsheet, the article including an absorbent composite disposed between the topsheet and the backsheet, the absorbent composite including a first intake layer including a resilient coform material, and a retention layer disposed between the topsheet and the backsheet, the retention layer including one of a coform material, a resilient coform material, an airlaid material, a high-density, hydrogen-bonded, fluff/superabsorbent polymer material, a spunlace material, a superabsorbent polymer/adhesive composite material, and a foam material.

The present disclosure also provides a method for making an absorbent personal care article having an absorbent composite, the method including merging a stream of an absorbent material with a stream of meltblown fibers to form a composite stream; collecting the composite stream on a forming surface to form a resilient coform nonwoven web; and combining the resilient coform nonwoven web with a topsheet and a backsheet.

The present disclosure also provides an absorbent composite adapted for use in an absorbent article having a topsheet and a backsheet, the absorbent composite including an intake layer including a foam material disposed between the topsheet and the backsheet, the intake layer having a plurality of holes therethrough, and a retention layer disposed between the topsheet and the backsheet, wherein the retention layer includes a resilient coform material.

Other features and aspects of the present disclosure are discussed in greater detail herein.

### BRIEF DESCRIPTION OF THE DRAWINGS

The foregoing and other features and aspects of the present disclosure and the manner of attaining them will become more apparent, and the disclosure itself will be better understood by reference to the following description, appended claims, and accompanying drawings.

FIG. 1 is a schematic illustration of one aspect of a method for forming the coform web of the present disclosure;

FIG. 2 is an illustration of certain features of the apparatus shown in FIG. 1;

FIG. 3 is a cross-sectional view of one aspect of a textured coform nonwoven web formed according to the present disclosure;

FIG. 4 is a photo of one aspect of a textured coform nonwoven web;

FIG. 5 is a photo of the textured coform nonwoven webs from FIG. 4 after being crumpled and allowed to relax;

FIG. 6 is a photo of another aspect of a textured coform nonwoven web;

FIG. 7 is a photo of the textured coform nonwoven webs from FIG. 6 after being crumpled and allowed to relax;

FIG. 8 is a schematic partially-cutaway plan view of a feminine hygiene article incorporating the absorbent composite of the present application;

FIG. 9 is a partial schematic side elevation of a feminine hygiene article incorporating the absorbent composite of the present application;

FIG. 10 is a plan view schematic of an example of a hole pattern on an absorbent composite outline used in testing an absorbent composite of the present application; and

FIG. 11 is a plan view schematic of an example of an absorbent composite outline used in testing an absorbent composite of the present application.

Repeat use of reference characters in the present specification and drawings is intended to represent the same or analogous features or elements of the present disclosure.

### DETAILED DESCRIPTION

It is to be understood by one of ordinary skill in the art that the present discussion is a description of exemplary aspects only, and is not intended as limiting the broader aspects of the present disclosure.

Reference now will be made in detail to various aspects of the disclosure, one or more examples of which are set forth below. Each example is provided by way of explanation, not limitation of the disclosure. In fact, it will be apparent to those skilled in the art that various modifications and variations can be made in the present disclosure without departing from the scope or spirit of the disclosure. For instance, features illustrated or described as part of one aspect, can be used on another aspect to yield a still further aspect. Thus, it is intended that the present disclosure cover such modifications and variations.

As used herein the term “nonwoven web” generally refers to a web having a structure of individual fibers or threads that are interlaid, but not in an identifiable manner as in a knitted fabric. Examples of nonwoven fabrics or webs include, but are not limited to, meltblown webs, spunbond webs, bonded carded webs, airlaid webs, coform webs, hydraulically entangled webs, and so forth.

As used herein, the term “spunbond web” generally refers to a web containing small diameter substantially continuous fibers. The fibers are formed by extruding a molten thermoplastic material from a plurality of fine, usually circular, capillaries of a spinnerette with the diameter of the extruded fibers then being rapidly reduced as by, for example, eductive drawing and/or other well-known spunbonding mechanisms. The production of spunbond webs is described and illustrated, for example, in U.S. Pat. Nos. 4,340,563 to Appel, et al., 3,692,618 to Dorschner, et al., 3,802,817 to Matsuki, et al., 3,338,992 to Kinney, 3,341,394 to Kinney, 3,502,763 to Hartman, 3,502,538 to Levy, 3,542,615 to Dobo, et al., and 5,382,400 to Pike, et al., which are incorporated herein in their entirety by reference thereto for all purposes. Spunbond fibers are generally not tacky when they are deposited onto a collecting surface. Spunbond fibers can sometimes have diameters less than about 40 micrometers, and are often between about 5 to about 20 micrometers.

Generally speaking, the present disclosure is directed to an absorbent composite having a resilient coform layer and optionally at least one or more additional layers. The resilient coform layer as described in more detail below is formed from a resilient coform nonwoven web that contains a matrix of meltblown fibers and an absorbent material. The absorbent composite can be used in a personal care or other suitable article.

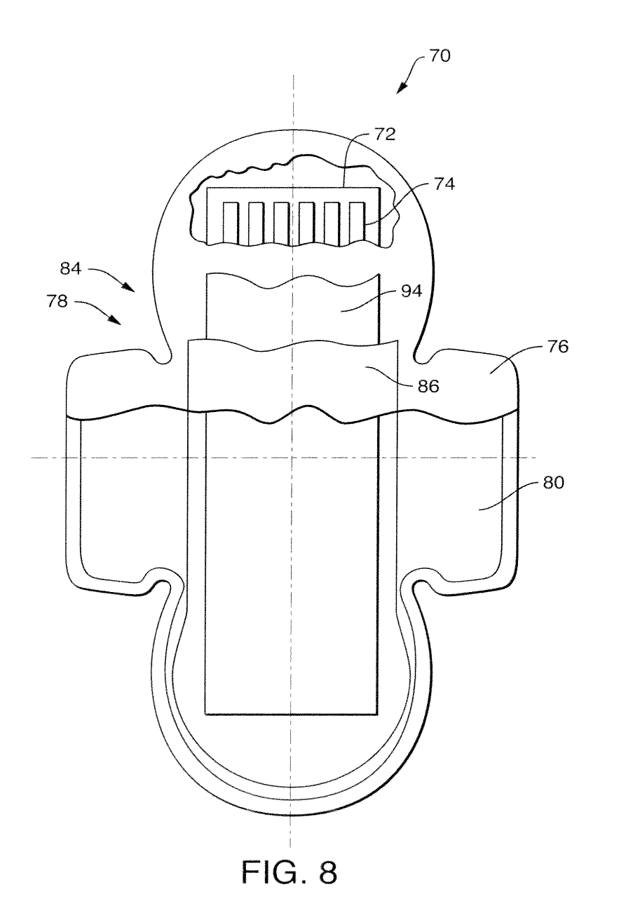

As one example, the absorbent composite can be used as an absorbent member in a feminine hygiene article. As shown in FIG. 8, a feminine hygiene article 70 includes a peel strip 72 that adhesively attaches by means of a garment attachment adhesive 74 to a garment-side backsheet 76 on one side. The other side of the backsheet 76 attaches to an absorbent layer 78 with construction adhesive. The absorbent layer 78 attaches to a body side liner or topsheet 80. The absorbent composite 84 of the present disclosure can suitably replace the absorbent layer 78. Desirably, use of the absorbent composite 84 will inhibit bunching of the product as it is worn, hence improving overall effectiveness and reducing leakage. Other suitable configurations for forming personal care articles with absorbent core materials are well known to those skilled in the art. In one desirable aspect, the absorbent composite 84 has a textured surface. The textured surface is desirably positioned towards the topsheet 80 to promote faster fluid intake and higher absorbency of the absorbent core.

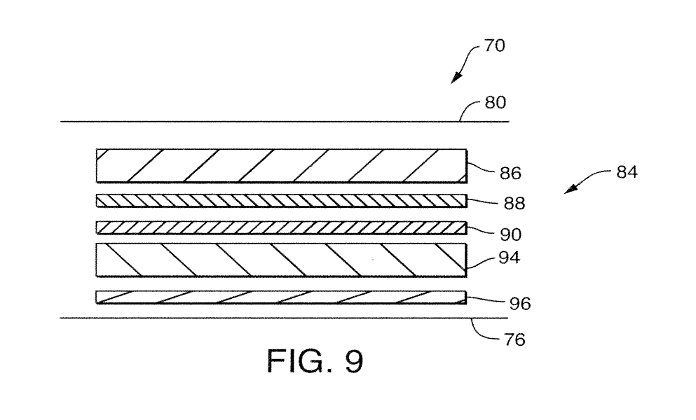

The absorbent composite 84 of the present disclosure, disposed between a topsheet 80 and a liquid-impermeable backsheet 76, includes one to three layers in addition to the resilient coform material. As illustrated in FIG. 9, the absorbent composite 84 can include an optional first liquid intake layer 86, an optional second liquid intake layer 88 or an optional first distribution layer 90, a retention layer 94, and an optional second distribution layer 96. The layers are generally disposed in a face-to-face orientation within the absorbent composite 84.

In various aspects of the present application, the first intake layer 86 can be wider and/or longer than the retention layer 94, and can be shaped other than rectangularly to better conform to the body while worn. In another aspect of the present application, the layer that includes resilient coform material can be the widest and/or longest layer. In yet another aspect of the present application, the layer that includes airlaid material can be the widest and/or longest layer.

At least one of the first intake and retention layers 86, 94 includes a resilient coform material that can function as a fluid intake material or as a fluid retention material, respectively. For the aspect in which the first intake layer 86 includes resilient coform material, the absorbent composite 84 includes an additional layer that can be a second liquid intake layer 88, a first distribution layer 90, a retention layer 94, or a second distribution layer 96. For the aspect in which the retention layer 94 includes resilient coform material, the absorbent composite 84 includes an additional layer that can be a first liquid intake layer 86, a second liquid intake layer 88, a first distribution layer 90, or a second distribution layer 96.

Materials included in each layer are described in more detail below. The first intake layer 86 can include a coform material, a resilient coform material, an airlaid material, a bonded-carded web (BCW) material, or a foam material, and further can include fluff pulp. The second liquid intake layer 88 can include a BCW material, an airlaid material, or a foam material. The first distribution layer 90 can include a spunlace material, a meltblown microfiber material, or a foam material. The retention layer 94 can include a coform material, a resilient coform material, or an airlaid material, each of which can further include a superabsorbent material (SAM). The retention layer 94 can instead include a high-density, hydrogen-bonded, fluff/superabsorbent polymer material, a spunlace material, a superabsorbent polymer/adhesive composite material, or a foam material. Any of these retention layer materials can further include fluff pulp. Finally, the second distribution layer 96 can include a meltblown microfiber material, a spunlace material, or a foam material, and can further include fluff pulp.

The absorbent composite 84 of the present disclosure can be used in a wide variety of articles. For example, the absorbent composite 84 can be incorporated into an absorbent article that is capable of absorbing water or other fluids. Examples of such absorbent articles include, but are not limited to, personal care absorbent articles, such as diapers, training pants, absorbent underpants, incontinence articles, feminine hygiene products (e.g., sanitary napkins), swim wear, baby wipes, mitt wipe, and so forth; medical absorbent articles, such as garments, fenestration materials, underpads, bed pads, bandages, absorbent drapes, and medical wipes; food service wipers; clothing articles; pouches; and so forth. Materials and processes suitable for forming such articles are well known to those skilled in the art. Several examples of such absorbent articles are described in U.S. Pat. Nos. 5,649,916 to DiPalma, et al.; 6,110,158 to Kielpikowski; 6,663,611 to Blaney, et al., which are incorporated herein in their entirety by reference thereto for all purposes. Still other suitable articles are described in U.S. Patent Application Publication No. 2004/0060112 A1 to Fell et al., as well as U.S. Pat. Nos. 4,886,512 to Damico et al.; 5,558,659 to Sherrod et al.; 6,888,044 to Fell et al.; and 6,511,465 to Freiburger et al., all of which are incorporated herein in their entirety by reference thereto for all purposes. When employed in the absorbent article, the absorbent composite 84 of the present disclosure can form a component of the absorbent core or any other absorbent component of the absorbent article as is well known in the art.

The term “coform” generally refers to a blend of meltblown fibers and absorbent fibers such as cellulosic fibers that can be formed by air forming a meltblown polymer material while simultaneously blowing air-suspended fibers into the stream of meltblown fibers. The coform material can also include other materials, such as superabsorbent materials. The meltblown fibers and absorbent fibers (and other optional materials) are collected on a forming surface, such as provided by a foraminous belt. The forming surface can include a gas-pervious material that has been placed onto the forming surface. Coform materials are further described in U.S. Pat. Nos. 5,508,102 and 5,350,624 to Georger et al. and 4,100,324 to Anderson, which are incorporated herein in their entirety by reference thereto to the extent they do not conflict herewith.

As used herein, the term “resilient coform” generally refers to a resilient coform nonwoven layer including a matrix of meltblown fibers and an absorbent material, wherein the meltblown fibers constitute from 30 wt % to about 99 wt % of the web and the absorbent material constitutes from about 1 wt % to about 70 wt % of the web, and further wherein the meltblown fibers being formed from a thermoplastic composition that contains at least one propylene/α-olefin copolymer having a propylene content of from about 60 mole % to about 99.5 mole % and an α-olefin content of from about 0.5 mole % to about 40 mole %, wherein the copolymer further has a density of from about 0.86 to about 0.90 grams per cubic centimeter and the composition has a melt flow rate of from about 120 to about 6000 grams per 10 minutes, determined at 230° C. in accordance with ASTM Test Method D1238-E, although practical considerations can reduce the high end melt flow rate range.

The meltblown fibers of the coform nonwoven web constitute from 30 wt % to about 99 wt % of the web and the absorbent material constitutes from about 1 wt % to about 70 wt % of the web. More preferably the meltblown fibers of the coform nonwoven web constitute from 45 wt % to about 99 wt % of the web and the absorbent material constitutes from about 1 wt % to about 55 wt % of the web. The meltblown fibers are formed from a thermoplastic composition described below that contains at least one propylene/α-olefin copolymer of a certain monomer content, density, melt flow rate, etc. The selection of a specific type of propylene/α-olefin copolymer provides the resulting composition with improved thermal properties for forming a coform web. For example, the thermoplastic composition crystallizes at a relatively slow rate, thereby allowing the fibers to remain slightly tacky during formation. This tackiness can provide a variety of benefits, such as enhancing the ability of the meltblown fibers to adhere to the absorbent material during web formation. The meltblown fibers can constitute from about 30 wt % to about 99 wt %, in particular aspects from about 45 wt % to about 99 wt %, in more particular aspects from about 50 wt % to about 90 wt %, and in even more particular aspects, from about 50 wt % to about 80 wt % of the coform web. Likewise, the absorbent material can constitute from about 1 wt % to about 70 wt %, in particular aspects 1 wt % to about 55 wt %, in more particular aspects from 10 wt % to about 50 wt %, and in even more particular aspects, from about 20 wt % to about 50 wt % of the coform web.

In addition to enhancing the bonding capacity of the meltblown fibers, the thermoplastic composition of the present disclosure can also impart other benefits to the resulting coform structure. In certain aspects, for example, the coform web can be imparted with texture using a three-dimensional forming surface. In such aspects, the relatively slow rate of crystallization of the meltblown fibers can increase their ability to conform to the contours of the three-dimensional forming surface. Once the fibers crystallize, however, the meltblown fibers can achieve a degree of resiliency greater than that of conventional polypropylene, thereby allowing them to both retain and regain the three-dimensional shape and highly textured surface on the coform web.

Another benefit of the fiber's prolonged tackiness during formation can be an increased ply attachment strength between layers of a multi-ply coform nonwoven web, resulting in additional shear energy being necessary to delaminate the plies. Such increased ply attachment strength can reduce or eliminate the need for embossing that could negatively impact sheet characteristics such as thickness and density. Increased ply attachment strength can be particularly desirable during dispensing of wipers made from a multi-ply coform nonwoven web. Texture imparted by using a three-dimensional forming surface as described herein can further increase the ply attachment strength by increasing the contact surface area between the plies.

Various aspects of the present disclosure will now be described in more detail.

The thermoplastic composition of the present disclosure contains at least one copolymer of propylene and an α-olefin, such as a C₂-C₂₀ α-olefin, C₂-C₁₂ α-olefin, or C₂-C₈ α-olefin. Suitable α-olefins can be linear or branched (e.g., one or more C₁-C₃ alkyl branches, or an aryl group). Specific examples include ethylene, butene; 3-methyl-1-butene; 3,3-dimethyl-1-butene; pentene; pentene with one or more methyl, ethyl or propyl substituents; hexene with one or more methyl, ethyl or propyl substituents; heptene with one or more methyl, ethyl or propyl substituents; octene with one or more methyl, ethyl or propyl substituents; nonene with one or more methyl, ethyl or propyl substituents; ethyl, methyl or dimethyl-substituted decene; dodecene; styrene; and so forth. Particularly desired α-olefin co-monomers are ethylene, butene (e.g., 1-butene), 7exene, and octene (e.g., 1-octene or 2-octene). The propylene content of such copolymers can be from about 60 mole % to about 99.5 mole %, in further aspects from about 80 mole % to about 99 mole %, and in even further aspects, from about 85 mole % to about 98 mole %. The α-olefin content can likewise range from about 0.5 mole % to about 40 mole %, in further aspects from about 1 mole % to about 20 mole %, and in even further aspects, from about 2 mole % to about 15 mole %. The distribution of the α-olefin co-monomer is typically random and uniform among the differing molecular weight fractions forming the propylene copolymer.

The density of the propylene/α-olefin copolymer can be a function of both the length and amount of the α-olefin. That is, the greater the length of the α-olefin and the greater the amount of α-olefin present, the lower the density of the copolymer. Generally speaking, copolymers with a higher density are better able to form a three-dimensional structure, while those with a lower density possess better elastomeric and resiliency properties. Thus, to achieve an optimum balance between texture and resiliency, the propylene/α-olefin copolymer is normally selected to have a density of about 0.860 grams per cubic centimeter (g/cm³) to about 0.900 g/cm³, in further aspects from about 0.861 to about 0.890 g/cm³, and in even further aspects, from about 0.862 g/cm³ to about 0.880 g/cm³. Further, the density of the thermoplastic composition is normally selected to have a density of about 0.860 grams per cubic centimeter (g/cm³) to about 0.940 g/cm³, in further aspects from about 0.861 to about 0.920 g/cm³, and in even further aspects, from about 0.862 g/cm³ to about 0.900 g/cm³.

Any of a variety of known techniques can generally be employed to form the propylene/α-olefin copolymer used in the meltblown fibers. For instance, olefin polymers can be formed using a free radical or a coordination catalyst (e.g., Ziegler-Natta). Preferably, the copolymer is formed from a single-site coordination catalyst, such as a metallocene catalyst. Such a catalyst system produces propylene copolymers in which the co-monomer is randomly distributed within a molecular chain and uniformly distributed across the different molecular weight fractions. Metallocene-catalyzed propylene copolymers are described, for instance, in U.S. Pat. Nos. 7,105,609 to Datta, et al.; 6,500,563 to Datta, et al.; 5,539,056 to Yang, et al.; and 5,596,052 to Resconi, et al., which are incorporated herein in their entirety by reference thereto for all purposes. Examples of metallocene catalysts include bis(n-butylcyclopentadienyl)titanium dichloride, bis(n-butylcyclopentadienyl)zirconium dichloride, bis(cyclopentadienyl)scandium chloride, bis(indenyl)zirconium dichloride, bis(methylcyclopentadienyl)titanium dichloride, bis(methylcyclopentadienyl)zirconium dichloride, cobaltocene, cyclopentadienyltitanium trichloride, ferrocene, hafnocene dichloride, isopropyl(cyclopentadienyl,-1-flourenyl)zirconium dichloride, molybdocene dichloride, nickelocene, niobocene dichloride, ruthenocene, titanocene dichloride, zirconocene chloride hydride, zirconocene dichloride, and so forth. Polymers made using metallocene catalysts typically have a narrow molecular weight range. For instance, metallocene-catalyzed polymers can have polydispersity numbers (M_{w}/Mₙ) of below 4, controlled short chain branching distribution, and controlled tacticity.

In particular aspects the propylene/α-olefin copolymer constitutes about 50 wt % or more, in further aspects about from 60 wt % or more, and in even further aspects, about 75 wt % or more of the thermoplastic composition used to form the meltblown fibers. In other aspects the propylene/α-olefin copolymer constitutes at least about 1 wt % and less than about 49 wt %, in particular aspects from at least about 1% and less than about 45 wt %, in further aspects from at least about 5% and less than about 45 wt %, and in even further aspects, from at least about 5 wt % and less than about 35 wt % of the thermoplastic composition used to form the meltblown fibers. Of course, other thermoplastic polymers can also be used to form the meltblown fibers so long as they do not adversely affect the desired properties of the composite. For example, the meltblown fibers can contain other polyolefins (e.g., polypropylene, polyethylene, etc.), polyesters, polyurethanes, polyamides, block copolymers, and so forth. In one aspect, the meltblown fibers can contain an additional propylene polymer, such as homo polypropylene or a copolymer of propylene. The additional propylene polymer can, for instance, be formed from a substantially isotactic polypropylene homopolymer or a copolymer containing equal to or less than about 10 weight percent of other monomer, i.e., at least about 90% by weight propylene. Such a polypropylene can be present in the form of a graft, random, or block copolymer and can be predominantly crystalline in that it has a sharp melting point above about 110° C., in further aspects about above 115° C., and in even further aspects, above about 130° C. Examples of such additional polypropylenes are described in U.S. Pat. No. 6,992,159 to Datta, et al., which is incorporated herein in its entirety by reference thereto for all purposes.

In particular aspects, additional polymer(s) can constitute from about 0.1 wt % to about 90 wt %, in further aspects from about 0.5 wt % to about 50 wt %, and in even further aspects, from about 1 wt % to about 30 wt % of the thermoplastic composition. Likewise, the above-described propylene/α-olefin copolymer can constitute from about 15 wt % to about 99.9 wt %, in further aspects from about 50 wt % to about 99.5 wt %, and in even further aspects, from about 70 wt % to about 99 wt % of the thermoplastic composition.

The thermoplastic composition used to form the meltblown fibers can also contain other additives as is known in the art, such as surfactants, melt stabilizers, processing stabilizers, heat stabilizers, light stabilizers, antioxidants, heat aging stabilizers, whitening agents, etc. Phosphite stabilizers (e.g., IRGAFOS available from Ciba Specialty Chemicals of Tarrytown, New York and DOVERPHOS available from Dover Chemical Corp. of Dover, Ohio) are exemplary melt stabilizers. In addition, hindered amine stabilizers (e.g., CHIMASSORB available from Ciba Specialty Chemicals) are exemplary heat and light stabilizers. Further, hindered phenols are commonly used as an antioxidant. Some suitable hindered phenols include those available from Ciba Specialty Chemicals (Ciba) of under the trade name IRGANOX, such as IRGANOX phenols 1076, 1010, or E 201. When employed, such additives (e.g., antioxidant, stabilizer, surfactants, etc.) can each be present in an amount from about 0.001 wt % to about 15 wt %, in further aspects, from about 0.005 wt % to about 10 wt %, and in even further aspects, from about 0.01 wt % to about 5 wt % of the thermoplastic composition used to form the meltblown fibers. One or more surfactants can be added to the polymer composition to make the polymer fibers more wettable and improve the fluid intake properties of the coform material. Suitable surfactants include cationic, anionic, amphoteric, and nonionic surfactants. A particularly suitable internal surfactant is available from Techmer PM, Clinto, Tennessee, is Hydrophilic Melt additive PPM15560 surfactant. When employed, the surfactants can each be present in an amount from about 0.5 wt % to about 10 wt %, in further aspects, from about 1.0 wt % to about 7.5 wt %, and in even further aspects, from about 1.5 wt % to about 5 wt % of the thermoplastic composition used to form the meltblown fibers. Surfactants can also be applied to the meltblown fibers externally as topical treatments.

Through the selection of certain polymers and their content, the resulting thermoplastic composition can possess thermal properties superior to polypropylene homopolymers conventionally employed in meltblown webs. For example, the thermoplastic composition is generally more amorphous in nature than polypropylene homopolymers conventionally employed in meltblown webs. For this reason, the rate of crystallization of the thermoplastic composition is slower, as measured by its “crystallization half-time”—i.e., the time required for one-half of the material to become crystalline. For example, the thermoplastic composition typically has a crystallization half-time of greater than about 5 minutes, in further aspects from about 5.25 minutes to about 20 minutes, and in even further aspects, from about 5.5 minutes to about 12 minutes, determined at a temperature of 125° C. To the contrary, conventional polypropylene homopolymers often have a crystallization half-time of 5 minutes or less. Further, the thermoplastic composition can have a melting temperature (“Tₘ”) of from about 100° C. to about 250° C., in further aspects from about 110° C. to about 200° C., and in even further aspects, from about 140° C. to about 180° C. The thermoplastic composition can also have a crystallization temperature (“T_{c}”) (determined at a cooling rate of 10° C./min) of from about 50° C. to about 150° C., in further aspects from about 80° C. to about 140° C., and in even further aspects, from about 100° C. to about 120° C. The crystallization half-time, melting temperature, and crystallization temperature can be determined using differential scanning calorimetry (“DSC”) as is well known to those skilled in the art.

The melt flow rate of the thermoplastic composition can also be selected within a certain range to optimize the properties of the resulting meltblown fibers. The melt flow rate is the weight of a polymer (in grams) that can be forced through an extrusion rheometer orifice (2.09 mm (0.0825 inch) diameter) when subjected to a force of 2160 grams in 10 minutes at 230° C. Generally speaking, the melt flow rate is high enough to improve melt processability, but not so high as to adversely interfere with the binding properties of the fibers to the absorbent material. Thus, in most aspects of the present disclosure, the thermoplastic composition has a melt flow rate of from about 120 to about 6000 grams per 10 minutes, in further aspects from about 150 to about 3000 grams per 10 minutes, and in even further aspects, from about 170 to about 1500 grams per 10 minutes, measured in accordance with ASTM Test Method D1238-E.

The term “meltblown fibers” refers to fibers formed by extruding a molten thermoplastic material through a plurality of fine, usually circular, die capillaries as molten threads or filaments into a high velocity, usually heated, gas (e.g., air) stream that attenuates the filaments of molten thermoplastic material to reduce their diameter. In the particular case of a coform process, the meltblown fiber stream intersects with one or more material streams that are introduced from a different direction. Thereafter, the meltblown fibers and other materials are carried by the high velocity gas stream and are deposited on a collecting surface. The distribution and orientation of the meltblown fibers within the formed web is dependent on the geometry and process conditions. Under certain process and equipment conditions, the resulting fibers can be substantially “continuous,” defined as having few separations, broken fibers or tapered ends when multiple fields of view are examined through a microscope at 10× or 20× magnification. When “continuous” melt blown fibers are produced, the sides of individual fibers will generally be parallel with minimal variation in fiber diameter within an individual fiber length. In contrast, under other conditions, the fibers can be overdrawn and strands can be broken and form a series of irregular, discrete fiber lengths and numerous broken ends. Retraction of the once attenuated broken fiber will often result in large clumps of polymer. Such a process is disclosed, for example, in U.S. Pat. No. 3,849,241 to Butin et al., which is hereby incorporated by reference in a manner that is consistent herewith.

The meltblown fibers can be monocomponent or multicomponent. Monocomponent fibers are generally formed from a polymer or blend of polymers extruded from a single extruder. Multicomponent fibers are generally formed from two or more polymers (e.g., bicomponent fibers) extruded from separate extruders. The polymers can be arranged in substantially constantly positioned distinct zones across the cross-section of the fibers. The components can be arranged in any desired configuration, such as sheath-core, side-by-side, pie, island-in-the-sea, three island, bull's eye, or various other arrangements known in the art. Various methods for forming multicomponent fibers are described in U.S. Patent Nos. 4,789,592 to Taniguchi et al., 5,336,552 to Strack et al., 5,108,820 to Kaneko, et al., 4,795,668 to Kruege, et al., 5,382,400 to Pike, et al., 5,336,552 to Strack, et al., and 6,200,669 to Marmon, et al., which are incorporated herein in their entirety by reference thereto for all purposes. Multicomponent fibers having various irregular shapes can also be formed, such as described in U.S. Pat. Nos. 5,277,976 to Hogle, et al., 5,162,074 to Hills, 5,466,410 to Hills, 5,069,970 to Largman, et al., and 5,057,368 to Largman, et al., which are incorporated herein in their entirety by reference thereto for all purposes. It should be noted that meltblown materials are typically treated with a wettability agent for applications such as those described herein. Any suitable wettability treatment can be used.

Any absorbent material can generally be employed in the coform nonwoven web, such as absorbent fibers, particles, etc. In one aspect, the absorbent material includes fibers formed by a variety of pulping processes, such as kraft pulp, sulfite pulp, thermomechanical pulp, etc. The pulp fibers can include softwood fibers having an average fiber length of greater than 1 mm and particularly from about 1.5 to 5 mm based on a length-weighted average. Such softwood fibers can include, but are not limited to, northern softwood, southern softwood, redwood, red cedar, hemlock, pine (e.g., southern pines), spruce (e.g., black spruce), combinations thereof, and so forth. Exemplary commercially available pulp fibers suitable for the present disclosure include those available from Weyerhaeuser Co. of Federal Way, Washington under the designation “CF-405.” Hardwood fibers, such as eucalyptus, maple, birch, aspen, and so forth, can also be used. In certain instances, eucalyptus fibers can be particularly desired to increase the softness of the web. Eucalyptus fibers can also enhance the brightness, increase the opacity, and change the pore structure of the web to increase its wicking ability. Moreover, if desired, secondary fibers obtained from recycled materials can be used, such as fiber pulp from sources such as, for example, newsprint, reclaimed paperboard, and office waste. Further, other natural fibers can also be used in the present disclosure, such as bamboo, abaca, sabai grass, milkweed floss, pineapple leaf, and so forth. In addition, in some instances, synthetic fibers can also be utilized.

Besides or in conjunction with pulp fibers, the absorbent material can also include a superabsorbent that is in the form of fibers, particles, gels, etc. Generally speaking, superabsorbents are water-swellable materials capable of absorbing at least about 10 times its weight and, in some cases, at least about 20 times or at least about 30 times its weight in an aqueous solution containing 0.9 weight percent sodium chloride. The superabsorbent can be formed from natural, synthetic and modified natural polymers and materials. Examples of synthetic superabsorbent polymers include materials including a lightly crosslinked unneutralized acidic water absorbing resin and a lightly crosslinked unneutralized basic water absorbing resin, as disclosed in U.S. Pat. No. 6,623,576 to Mitchell et al. Additionally, examples include the alkali metal and ammonium salts of poly(acrylic acid) and poly(methacrylic acid), poly(acrylamides), poly(vinyl ethers), maleic anhydride copolymers with vinyl ethers and alpha-olefins, poly(vinyl pyrrolidone), poly(vinylmorpholinone), poly(vinyl alcohol), and mixtures and copolymers thereof. Further, superabsorbents include natural and modified natural polymers, such as starch, hydrolyzed acrylonitrile-grafted starch, acrylic acid grafted starch, methyl cellulose, chitosan, carboxymethyl cellulose, hydroxypropyl cellulose, and the natural gums, such as alginates, xanthan gum, locust bean gum and so forth. Mixtures of natural and wholly or partially synthetic superabsorbent polymers can also be useful in the present disclosure. Particularly suitable superabsorbent polymers are HYSORB 8760 superabsorbent (available from BASF of Charlotte, N.C.) and FAVOR SXM 9500 superabsorbent (available from EVONIK Stockhausen of Greensboro, North Carolina).

The coform web of the present disclosure is generally made by a process in which at least one meltblown die head (e.g., two) is arranged near a chute through which the absorbent material is added while the web forms. Some examples of such coform techniques are disclosed in U.S. Pat. Nos. 4,100,324 to Anderson, et al.; 5,350,624 to Georger, et al.; and 5,508,102 to Georger, et al., as well as U.S. Patent Application Publication Nos. 2003/0200991 to Keck, et al. and 2007/0049153 to Dunbar, et al., all of which are incorporated herein in their entirety by reference thereto for all purposes.

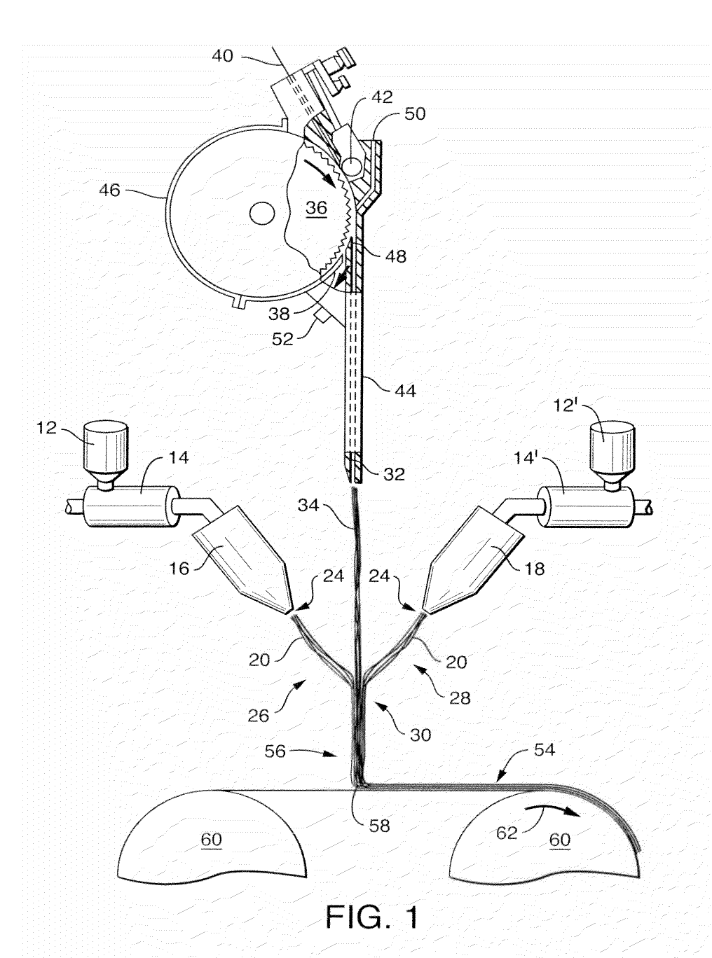

Referring to FIG. 1, for example, one aspect of an apparatus is shown for forming a coform web of the present disclosure. In this aspect, the apparatus includes a pellet hopper 12 or 12′ of an extruder 14 or 14′, respectively, into which a propylene/α-olefin thermoplastic composition can be introduced. The extruders 14 and 14′ each have an extrusion screw (not shown), which is driven by a conventional drive motor (not shown). As the polymer advances through the extruders 14 and 14′, it is progressively heated to a molten state due to rotation of the extrusion screw by the drive motor. Heating can be accomplished in a plurality of discrete steps with its temperature being gradually elevated as it advances through discrete heating zones of the extruders 14 and 14′ toward two meltblowing dies 16 and 18, respectively. The meltblowing dies 16 and 18 can be yet another heating zone where the temperature of the thermoplastic resin is maintained at an elevated level for extrusion.

When two or more meltblowing die heads are used, such as described above, it should be understood that the fibers produced from the individual die heads can be different types of fibers. That is, one or more of the size, shape, or polymeric composition can differ, and furthermore the fibers can be monocomponent or multicomponent fibers. For example, larger fibers can be produced by the first meltblowing die head, such as those having an average diameter of about 10 micrometers or more, in further aspects about 15 micrometers or more, and in even further aspects, from about 20 to about 50 micrometers, while smaller fibers can be produced by the second die head, such as those having an average diameter of about 10 micrometers or less, in further aspects about 7 micrometers or less, and in even further aspects, from about 2 to about 6 micrometers. In addition, it can be desirable that each die head extrude approximately the same amount of polymer such that the relative percentage of the basis weight of the coform nonwoven web material resulting from each meltblowing die head is substantially the same. Alternatively, it can also be desirable to have the relative basis weight production skewed, such that one die head or the other is responsible for the majority of the coform web in terms of basis weight. As a specific example, for a meltblown fibrous nonwoven web material having a basis weight of 1.0 ounces per square yard or “osy” (34 grams per square meter or “gsm”), it can be desirable for the first meltblowing die head to produce about 30 percent of the basis weight of the meltblown fibrous nonwoven web material, while one or more subsequent meltblowing die heads produce the remainder 70 percent of the basis weight of the meltblown fibrous nonwoven web material. Generally speaking, the overall basis weight of the coform nonwoven web is from about 10 gsm to about 350 gsm, and more particularly from about 17 gsm to about 200 gsm, and still more particularly from about 25 gsm to about 150 gsm.

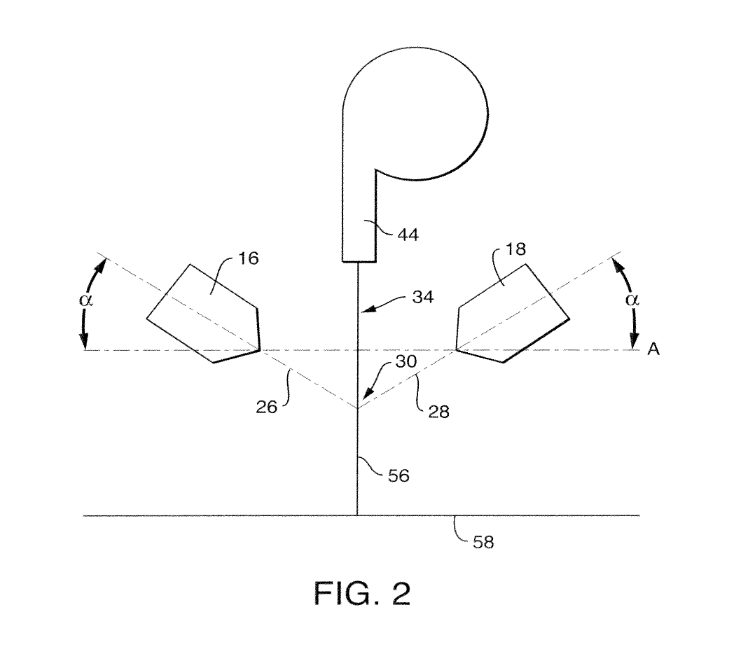

Each meltblowing die 16 and 18 is configured so that two streams of attenuating gas per die converge to form a single stream of gas that entrains and attenuates molten threads 20 as they exit small holes or orifices 24 in each meltblowing die. The molten threads 20 are formed into fibers or, depending upon the degree of attenuation, microfibers, of a small diameter that is usually less than the diameter of the orifices 24. Thus, each meltblowing die 16 and 18 has a corresponding single stream of gas 26 and 28 containing entrained thermoplastic polymer fibers. The gas streams 26 and 28 containing polymer fibers are aligned to converge at an impingement zone 30. Typically, the meltblowing die heads 16 and 18 are arranged at a certain angle with respect to the forming surface, such as described in U.S. Pat. Nos. 5,508,102 and 5,350,624 to Georger et al. Referring to FIG. 2, for example, the meltblown dies 16 and 18 can be oriented at an angle α as measured from a plane “A” tangent to the two dies 16 and 18. As shown, the plane “A” is generally parallel to the forming surface 58 (FIG. 1). Typically, each die 16 and 18 is set at an angle ranging from about 30 to about 75 degrees, in further aspects from about 35° to about 60°, and in even further aspects from about 45° to about 55°. The dies 16 and 18 can be oriented at the same or different angles. In fact, the texture of the coform web can actually be enhanced by orienting one die at an angle different than another die.

Referring again to FIG. 1, absorbent fibers 32 (e.g., fluff pulp fibers) are added to the two streams 26 and 28 of thermoplastic polymer fibers 20 and 21, respectively, and at the impingement zone 30. Introduction of the absorbent fibers 32 into the two streams 26 and 28 of thermoplastic polymer fibers 20 and 21, respectively, is designed to produce a graduated distribution of absorbent fibers 32 within the combined streams 26 and 28 of thermoplastic polymer fibers. This can be accomplished by merging a secondary gas stream 34 containing the absorbent fibers 32 between the two streams 26 and 28 of thermoplastic polymer fibers 20 and 21 so that all three gas streams converge in a controlled manner. Because they remain relatively tacky and semi-molten after formation, the meltblown fibers 20 and 21 can simultaneously adhere and entangle with the absorbent fibers 32 upon contact therewith to form a coherent nonwoven structure.

To accomplish the merger of the fibers, any conventional equipment can be employed, such as a picker roll 36 having a plurality of teeth 38 adapted to separate a mat or batt 40 of absorbent fibers into the individual absorbent fibers 32. When employed, the sheets or mats 40 of fibers 32 are fed to the picker roll 36 by a roller arrangement 42. After the teeth 38 of the picker roll 36 have separated the mat 40 of fibers 32 into separate absorbent fibers 32, the individual fibers 32 are conveyed toward the stream of thermoplastic polymer fibers through a nozzle 44. A housing 46 encloses the picker roll 36 and provides a passageway or gap 48 between the housing 46 and the surface of the teeth 38 of the picker roll 36. A gas, for example, air, is supplied to the passageway or gap 48 between the surface of the picker roll 36 and the housing 46 by way of a gas duct 50. The gas duct 50 can enter the passageway or gap 48 at the junction 52 of the nozzle 44 and the gap 48. The gas is supplied in sufficient quantity to serve as a medium for conveying the absorbent fibers 32 through the nozzle 44. The gas supplied from the duct 50 also serves as an aid in removing the absorbent fibers 32 from the teeth 38 of the picker roll 36. The gas can be supplied by any conventional arrangement such as, for example, an air blower (not shown). It is contemplated that additives and/or other materials can be added to or entrained in the gas stream to treat the absorbent fibers 32. The individual absorbent fibers 32 are typically conveyed through the nozzle 44 at about the velocity at which the absorbent fibers 32 leave the teeth 38 of the picker roll 36. In other words, the absorbent fibers 32, upon leaving the teeth 38 of the picker roll 36 and entering the nozzle 44, generally maintain their velocity in both magnitude and direction from the point where they left the teeth 38 of the picker roll 36. Such an arrangement is discussed in more detail in U.S. Pat. No. 4,100,324 to Anderson, et al.

If desired, the velocity of the secondary gas stream 34 can be adjusted to achieve coform structures of different properties. For example, when the velocity of the secondary gas stream 34 is adjusted so that it is greater than the velocity of each stream 26 and 28 of thermoplastic polymer fibers 20 and 21 upon contact at the impingement zone 30, the absorbent fibers 32 are incorporated in the coform nonwoven web 54 in a gradient structure. That is, the absorbent fibers 32 have a higher concentration between the outer surfaces of the coform nonwoven web 54 than at the outer surfaces. On the other hand, when the velocity of the secondary gas stream 34 is less than the velocity of each stream 26 and 28 of thermoplastic polymer fibers 20 and 21 upon contact at the impingement zone 30, the absorbent fibers 32 are incorporated in the coform nonwoven web 54 in a substantially homogenous fashion. That is, the concentration of the absorbent fibers 32 is substantially the same throughout the coform nonwoven web 54. This is because the low-speed stream of absorbent fibers 32 is drawn into a high-speed stream of thermoplastic polymer fibers 20, 21 to enhance turbulent mixing, which results in a consistent distribution of the absorbent fibers 32.

To convert the composite stream 56 of thermoplastic polymer fibers 20, 21 and absorbent fibers 32 into a coform nonwoven structure 54, a collecting device is located in the path of the composite stream 56. The collecting device can be a forming surface 58 (e.g., belt, drum, wire, fabric, etc.) driven by rollers 60 and that rotates as indicated by the arrow 62 in FIG. 1. The merged streams of thermoplastic polymer fibers 20, 21 and absorbent fibers 32 are collected as a coherent matrix of fibers on the surface of the forming surface 58 to form the coform nonwoven web 54. If desired, a vacuum box (not shown) can be employed to assist in drawing the near-molten meltblown fibers onto the forming surface 58. The resulting textured coform structure 54 is coherent and can be removed from the forming surface 58 as a self-supporting nonwoven material.

It should be understood that the present disclosure is by no means limited to the above-described aspects. In an alternative aspect, for example, first and second meltblowing die heads can be employed that extend substantially across a forming surface in a direction that is substantially transverse to the direction of movement of the forming surface. The die heads can likewise be arranged in a substantially vertical disposition, i.e., perpendicular to the forming surface, so that the thus-produced meltblown fibers are blown directly down onto the forming surface. Such a configuration is well known in the art and described in more detail in, for instance, U.S. Patent Application Publication No. 2007/0049153 to Dunbar, et al. Furthermore, although the above-described aspects employ multiple meltblowing die heads to produce fibers of differing sizes, a single die head can also be employed. An example of such a process is described, for instance, in U.S. Patent Application Publication No. 2005/0136781 to Lassig, et al., which is incorporated herein in its entirety by reference thereto for all purposes.

As indicated above, it is desired in certain cases to form a coform web that is textured. Referring again to FIG. 1, for example, one aspect of the present disclosure employs a forming surface 58 that is foraminous in nature so that the fibers can be drawn through the openings of the surface and form dimensional cloth-like tufts projecting from the surfaces of the material that correspond to the openings in the forming surface 58. The foraminous surface can be provided by any material that provides sufficient openings for penetration by some of the fibers, such as a highly permeable forming wire. Wire weave geometry and processing conditions can be used to alter the texture or tufts of the material. The particular choice will depend on the desired peak size, shape, depth, surface tuft “density” (that is, the number of peaks or tufts per unit area), etc. In one aspect, for example, the wire can have an open area of from about 35% and about 65%, in further aspects from about 40% to about 60%, and in even further aspects, from about 45% to about 55%. One exemplary high open area forming surface is the FORMTECH 6 forming wire manufactured by Albany International Co. of Albany, New York. Such a wire has a “mesh count” of about six strands by six strands per square inch (about 2.4 by 2.4 strands per square centimeter), i.e., resulting in about 36 foramina or “holes” per square inch (about 5.6 per square centimeter), and therefore capable of forming about 36 tufts or peaks in the material per square inch (about 5.6 peaks per square centimeter). The FORMTECH 6 forming wire also has a warp diameter of about 1 millimeter polyester, a shute diameter of about 1.07 millimeters polyester, a nominal air permeability of approximately 41.8 m³/min (1475 ft³/min), a nominal caliper of about 0.2 centimeters (0.08 inch) and an open area of approximately 51%. Another exemplary forming surface available from the Albany International Co. is the FORMTECH 10 forming wire, which has a mesh count of about 10 strands by 10 strands per square inch (about 4 by 4 strands per square centimeter), i.e., resulting in about 100 foramina or “holes” per square inch (about 15.5 per square centimeter), and therefore capable of forming about 100 tufts or peaks per square inch (about 15.5 peaks per square centimeter) in the material. Still another suitable forming wire is FORMTECH 8 forming wire, which has an open area of 47% and is also available from Albany International. Of course, other forming wires and surfaces (e.g., drums, plates, mats, etc.) can be employed. For examples, mats can be used with depressions engraved in the surface such that the coform fibers will fill the depressions to result in tufts that correspond with the depressions. The depressions (tufts) can take on various shapes, including, but not limited to, circles, squares, rectangles, swirls, ribs, lines, clouds, and so forth. Also, surface variations can include, but are not limited to, alternate weave patterns, alternate strand dimensions, release coatings (e.g., silicones, fluorochemicals, etc.), static dissipation treatments, and the like. Still other suitable foraminous surfaces that can be employed are described in U.S. Patent Application Publication No. 2007/0049153 to Dunbar, et al.

Regardless of the particular texturing method employed, the tufts formed by the meltblown fibers of the present disclosure are better able to retain the desired shape and surface contour. Namely, because the meltblown fibers crystallize at a relatively slow rate, they are soft upon deposition onto the forming surface, which allows them to drape over and conform to the contours of the surface. After the fibers crystallize, they are then able to hold the shape and form tufts. The size and shape of the resulting tufts depends upon the type of forming surface used, the types of fibers deposited thereon, the volume of below wire air vacuum used to draw the fibers onto and into the forming surface, and other related factors. For example, the tufts can project from the surface of the material in the range of about 0.25 millimeters to at least about 9 millimeters, and in further aspects, from about 0.5 millimeters to about 3 millimeters. Generally speaking, the tufts are filled with fibers and thus have desirable resiliency useful for wiping and scrubbing.

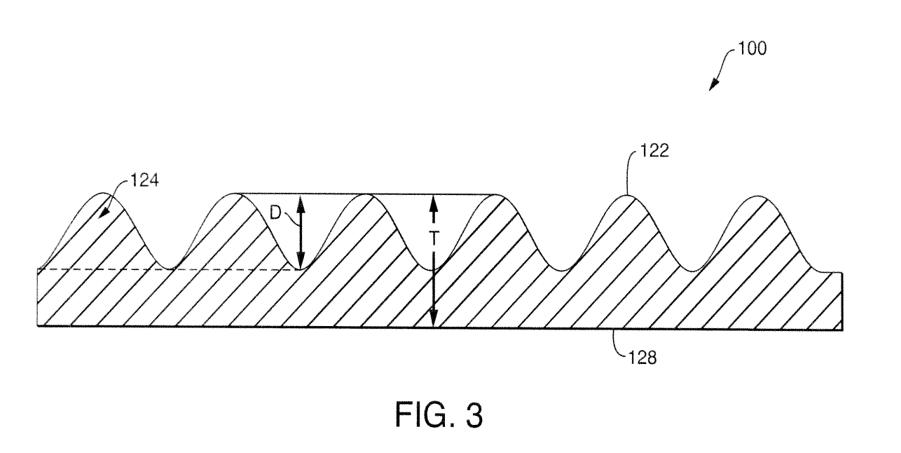

FIG. 3 shows an illustration of a cross section of a textured coform web 100 having a first exterior surface 122 and a second exterior surface 128. At least one of the exterior surfaces 122, 128 has a three-dimensional surface texture. In FIG. 3, for instance, the first exterior surface 122 has a three-dimensional surface texture that includes tufts or peaks 124 extending upwardly from the plane of the coform material. One indication of the magnitude of three-dimensionality in the textured exterior surface(s) of the coform web 100 is the peak to valley ratio, which is calculated as the ratio of the overall thickness “T” divided by the valley depth “D.” When textured in accordance with the present disclosure, the coform web 100 typically has a peak to valley ratio of about 5 or less, in further aspects from about 0.1 to about 4, and in even further aspects, from about 0.5 to about 3. The number and arrangement of the tufts 24 can vary widely depending on the desired end use. In particular aspects that are more densely textured, the textured coform web 100 will have from about 2 and about 70 tufts 24 per square centimeter, and in other aspects, from about 5 and 50 tufts 24 per square centimeter. In certain aspects that are less densely textured, the textured coform web 100 will have from about 100 to about 20,000 tufts per square meter, and in further aspects will have from about 200 to about 10,000 tufts per square meter. The textured coform web 100 can also exhibit a three-dimensional texture on the second surface of the web 120. This will especially be the case for lower basis weight materials, such as those having a basis weight of less than about 70 grams per square meter due to “mirroring”, wherein the second surface of the material exhibits peaks offset or between peaks on the first exterior surface 122 of the material. In this case, the valley depth D is measured for both exterior surfaces 122, 128 as above and are then added together to determine an overall material valley depth.

The coform material 100 of the present disclosure can be better understood with reference to the following coform test methods and examples.

### Coform Test Methods

### Melt Flow Rate:

The melt flow rate (“MFR”) is the weight of a polymer (in grams) forced through an extrusion rheometer orifice (2.09 mm (0.0825 inch) diameter) when subjected to a load of 2160 grams in 10 minutes at 230° C. Unless otherwise indicated, the melt flow rate was measured in accordance with ASTM Test Method D1238-E.

### Thermal Properties:

The melting temperature and crystallization temperature were determined by differential scanning calorimetry (DSC) in accordance with ASTM D-3417. The differential scanning calorimeter was a DSC Q100 Differential Scanning calorimeter that was outfitted with a liquid nitrogen cooling accessory and with a UNIVERSAL ANALYSIS 2000 (version 4.6.6) analysis software program, both of which are available from T.A. Instruments Inc. of New Castle, Delaware. To avoid directly handling the samples, tweezers or other tools were used. The samples were placed into an aluminum pan and weighed to an accuracy of 0.01 milligram on an analytical balance. A lid was crimped over the material sample onto the pan. Typically, the resin pellets were placed directly in the weighing pan, and the fibers were cut to accommodate placement on the weighing pan and covering by the lid.

The differential scanning calorimeter was calibrated using an indium metal standard and a baseline correction was performed, as described in the operating manual for the differential scanning calorimeter. A material sample was placed into the test chamber of the differential scanning calorimeter for testing, and an empty pan is used as a reference. All testing was run with a 55-cubic centimeter per minute nitrogen (industrial grade) purge on the test chamber. For resin pellet samples, the heating and cooling program was a 2-cycle test that began with an equilibration of the chamber to −25° C., followed by a first heating period at a heating rate of 10° C. per minute to a temperature of 200° C., followed by equilibration of the sample at 200° C. for 3 minutes, followed by a first cooling period at a cooling rate of 10° C. per minute to a temperature of −25° C., followed by equilibration of the sample at −25° C. for 3 minutes, and then a second heating period at a heating rate of 10° C. per minute to a temperature of 200° C. All testing was run with a 55-cubic centimeter per minute nitrogen (industrial grade) purge on the test chamber. The results were then evaluated using the UNIVERSAL ANALYSIS 2000 analysis software program that identified and quantified the melting and crystallization temperatures.

### Coform Examples

Various samples of coform webs were formed from two heated streams of meltblown fibers and a single stream of fiberized pulp fibers as described above and shown in FIG. 1. In various samples, the meltblown fibers were formed from the following polymer compositions:

1. The Example 1 polymer composition was a propylene homopolymer having a density of 0.91 g/cm³, a melt flow rate of 1200 g/10 minute (230° C., 2.16 kg) a crystallization temperature of 113° C., and a melting temperature of 156° C., which is available as METOCENE MF650X polymer from LyondellBasell Industries in Rotterdam, The Netherlands.

2. The Example 2 polymer composition was a blend of 75 wt % propylene homopolymer (ACHIEVE 6936G1 polymer) and 25 wt % propylene/ethylene copolymer (VISTAMAXX 2370 polymer, density 0.868 g/cm³, meltflow rate of 200 g/10 minutes (230° C., 2.16 kg)) having a density of 0.89 g/cm³ and a melt flow rate of 540 g/10 minutes (230° C., 2.16 kg), which are available from ExxonMobil Chemical Corp. of Houston, Tex.

3. The Example 3 polymer composition was an olefinic based elastomer (VISTAMAXX 2330 polymer, density 0.868 g/cm³, meltflow rate of 290 g/10 minutes (230° C., 2.16 kg), ethylene content 13.0 wt %), which is available from ExxonMobil Chemical Corp. of Houston, Tex.

The polymer compositions each further contained 3.0 wt % of surfactant (IRGASURF HL 560 surfactant, available from Ciba/BASF of Charlotte, N.C.). The pulp fibers were fully treated southern softwood pulp obtained from the Weyerhaeuser Co. of Federal Way, Washington under the designation “CF-405.”

For each Example, the polymer for each meltblown fiber stream was supplied to respective meltblown dies at a rate of 2.0 pounds of polymer per inch of die tip per hour through 0.020 inch diameter holes to achieve a meltblown fiber content of 50 wt %. The distance from the impingement zone to the forming wire (i.e., the forming height) was approximately 12 inches and the distance between the tips of the meltblown dies was approximately 6 inches. The meltblown die positioned upstream from the pulp fiber stream was oriented at an angle of 48° relative to the pulp stream, while the other meltblown die (positioned downstream from the pulp stream) was oriented at an angle of 48° relative to the pulp stream. The forming wire was FORMTECH 8 forming wire (Albany International Corp. of Albany, New York). To achieve different types of tufts, rubber mats were disposed on the upper surface of the forming wire. One such mat had a thickness of approximately 0.95 centimeters and contained holes arranged in a hexagonal array. The holes had a diameter of approximately 0.64 centimeters and were spaced apart approximately 0.95 centimeters (center-to-center). Mats of other patterns (e.g., clouds) were also used. A vacuum box was positioned below the forming wire to aid in deposition of the web and was set to 30 inches of water.

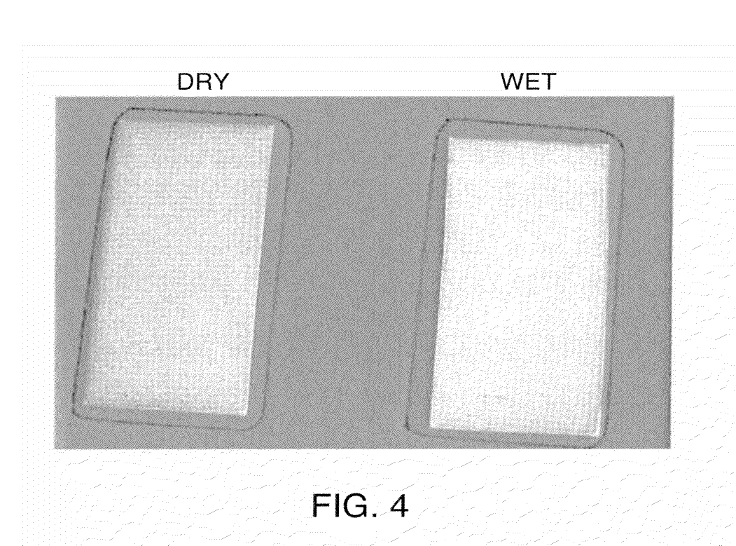

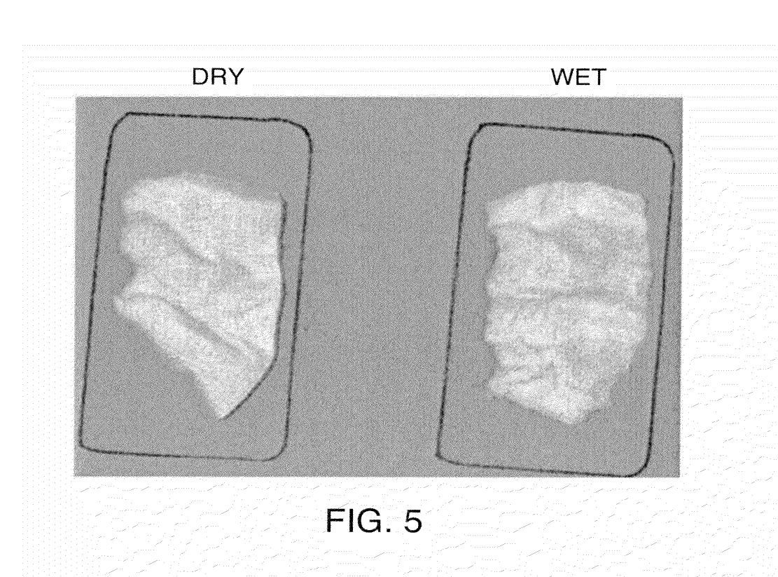

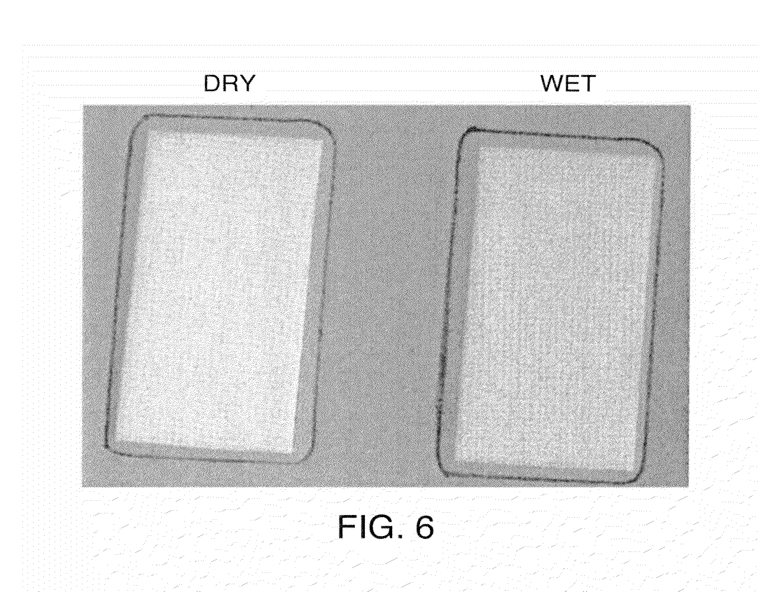

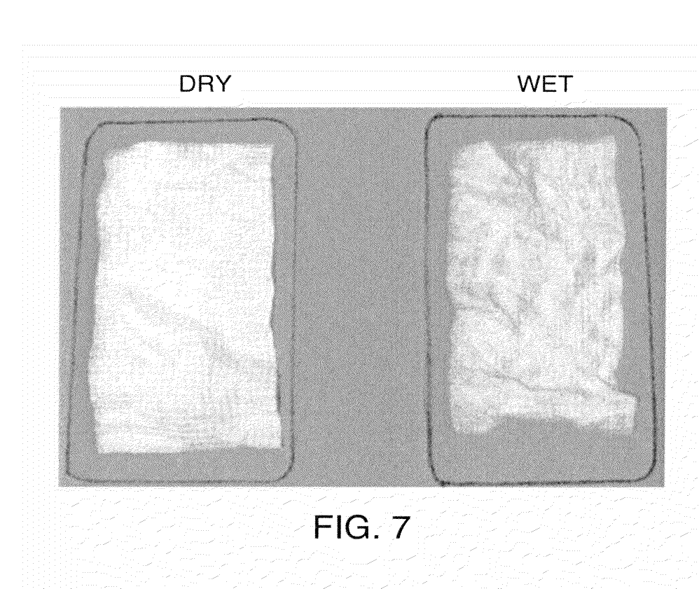

To demonstrate the resilient nature of the coform webs, samples of each Example were subjected to a “crumple” test. Each sample was three inches by seven inches. The test was done on both dry and wet samples. The wet samples had 3× its weight in water added to the sample. Each sample was compressed by balling it lightly into a tester's hand where the sample was held for 10 seconds. The samples were then released, lightly shaken out, and laid on a board. The samples were not subsequently smoothed in any way. FIG. 4 shows a photo of Example 1 samples prior to crumpling. FIG. 5 shows a photo of Example 1 samples after completion of the crumple test. FIG. 6 shows a photo of Example 3 samples prior to crumpling. FIG. 7 shows a photo of Example 3 samples after completion of the crumple test. As can be seen in FIGS. 4-7 the Example 3 samples were much more resilient, i.e., opened out much flatter after the crumple test, than Example 1. It was likewise found that the Example 2 samples behaved similar to the Example 3 samples.

In another aspect of the present disclosure, one of the layers of the absorbent composite 84 can include an airlaid material. The airlaid material, if in the retention layer 94, can also include superabsorbent material of the kinds described above for the coform layer, including superabsorbent polymer particles or superabsorbent polymer fibers. Airlaid material in any layer can also include fluff pulp fibers of the kinds described above for the coform layer. Commercially-available airlaid materials include those from Concert Gatineau of Gatineau, Quebec, Canada. Airlaid materials are combinations of fluff pulp and binder fibers that are heated to melt the binder fiber to the fluff pulp resulting in a stabilized structure.

The airlaid material can be constructed of a blend of a first group of fibers, a binder preferably in the form of a second group of fibers, and can further include a superabsorbent material. The combination is cured to form a stabilized, airlaid absorbent structure. The airlaid material in this aspect can have a predetermined basis weight of from between about 50 gsm to about 600 gsm. Preferably, the airlaid material has a basis weight of from between about 100 gsm to about 400 gsm. Most preferably, the airlaid material has a basis weight of about 200 gsm. The first group of fibers can be cellulosic fibers, such as fluff pulp fibers, that are short in length, have a high denier, and are hydrophilic. The first group of fibers can be formed from 100% softwood fibers. Preferably, the first group of fibers is southern pine Kraft pulp fibers having a length of about 2.5 mm and a denier of greater than 2.0. The denier of cellulosic fibers can be determined by running a coarseness test on a Kajanni analyzer to obtain a coarseness value in the units of milligrams per 100 meters (mg/100 m). This coarseness value is then divided by a constant value 11.1 to obtain a common textile denier in the units of grams per 9000 meters (g/9000 m). Suitable materials to use for the first group of fibers include Weyerhaeuser NB 416 pulp fibers and CF 405 partially-treated fluff pulp fibers that are commercially available from Weyerhaeuser Company of Federal Way, Washington, and Golden Isles 4881 and 4825 fluff pulp fibers that are commercially available from Georgia Pacific of Atlanta, Ga., although any suitable fluff pulp fibers can be used.

The binder portion of the retention layer can be a chemical coating. Preferably, the binder portion of the retention layer will include a second group of fibers. The second group of fibers can be synthetic binder fibers. Synthetic binder fibers are commercially available from several suppliers including Fibervisions Incorporated of Athens, Ga. and Fibervisions a/s of Varde, Denmark. Other suppliers of binder fibers are Huvis Corporation of South Korea and Far Eastern Textile Company Ltd. of Taiwan. Preferably, the second group of fibers is bicomponent fibers having a polyester core surrounded by a polyethylene sheath. Alternatively, the second group of fibers can be bicomponent fibers having a polypropylene core surrounded by a polyethylene sheath. In alternative aspects, airlaid that is made of different types of these synthetic binder fibers can be used.

The fibers making up the second group of fibers are typically longer in length and have a lower denier than the fibers making up the first group of fibers. The length of the fibers of the second group of fibers can range from between about 3 mm to about 6 mm. A fiber length of 3 mm works well. The fibers of the second group of fibers can have a denier of less than or equal to 2.0. The second group of fibers should be moisture insensitive and can be either crimped or non-crimped. Crimped fibers are preferred because they process better.

The airlaid material can also contain a superabsorbent material. A superabsorbent material is a material that is capable of absorbing at least 10 grams of water per gram of superabsorbent material. The superabsorbent material is preferably in the shape of small particles, although fibers, flakes, or other forms of superabsorbent materials can also be used. A suitable superabsorbent material is FAVOR SXM 9500 superabsorbent available from EVONIK Stockhausen, Inc. of Greensboro, North Carolina. Other similar types of superabsorbent materials, some of which are commercially available from BASF of Charlotte, N.C., such as HYSORB 8760 superabsorbent, can also be used. Preferably, the superabsorbent material is present in a weight percent of from between about 5% to about 60%.

The individual components of the airlaid material can be present in varying amounts. In addition, components can be distributed homogeneously or heterogeneously throughout the airlaid. It has been found, however, that the following percentages work well in forming a thin absorbent article. The first group of fibers can range from between about 30% to about 85%, by weight, of the airlaid material. The second group of fibers can range from between about 5% to about 20%, by weight, of the airlaid material. The superabsorbent material can range from between about 5% to about 60%, by weight, of the airlaid material. It has been found that forming an airlaid material with about 77% of the first group of fibers, about 8% of the second group of fibers, and about 15% of superabsorbent material works well for absorbing and retaining urine or menses.

The first group of fibers can be present in the airlaid material by a greater percent, by weight, than the second group of fibers. By using a greater percent of the first group of fibers, one can reduce the overall cost of the airlaid material. The first group of fibers also ensures that an absorbent article has sufficient fluid absorbing capacity. Cellulosic fibers such as fluff pulp fibers are generally less expensive than synthetic binder fibers. For good performance, the second group of fibers can make up at least about 5% of the airlaid material, by weight, to ensure that the airlaid material has sufficient tensile strength. As stated above, the airlaid material should be a mixture of the components.

In one aspect of the present disclosure, the airlaid material is compressed in a substantially dry condition after heat curing at a temperature of about 165 degrees Celsius for a time of from between about 8 seconds to about 10 seconds to a density ranging from between about 0.05 grams per cubic centimeter g/cm³ to about 0.3 g/cm³. Preferably, the airlaid material is compressed in a substantially dry condition to a density ranging from between about 0.07 g/cm³ to about 0.22 g/cm³. Most preferably, the airlaid material is compressed in a substantially dry condition to a density of at about 0.12 (g/cm³). This compression of the airlaid material will assist in forming a thin absorbent article.

Airlaid material, when used in an intake layer, typically does not include superabsorbent material, and has a density ranging from 0.05 g/cm³ to 0.15 g/cm³. Airlaid material used in a retention layer typically includes superabsorbent material and has a density ranging from 0.1 g/cm³ to 0.3 g/cm³.

It should be noted that the stabilized material making up the airlaid material should have sufficient tensile strength in the machine direction to allow winding it into rolls that can later be unwound and processed on converting equipment. Sufficient tensile strength can be achieved by varying the content of the binder fiber, adjusting the curing conditions, changing the specific density to which the fibers are compacted, as well as other ways known to one skilled in the art. It has been found that the airlaid material should have a tensile strength of at least 12 Newtons per 50 mm (N/50 mm). Preferably, the airlaid material should have a tensile strength of at least 18 N/50 mm. More preferably, the airlaid material should have a needed tensile strength of at least 25 N/50 mm. The tensile strength of the material can be tested using a tester such as a Model MTS/Sintech 1/S tester that is commercially sold by MTS Systems Corporation of Research Triangle Park, North Carolina. The tensile strength at peak load for the purpose of this disclosure is measured by securing a 50 mm strip of stabilized material between two movable jaws of a tensile tester. A distance of about 10 cm initially separates the two jaws. The two jaws are then moved outward away from one another at a rate of 25 cm/minute until the strip of material breaks. The tensile strength is recorded as peak load.

In another aspect of the present disclosure, the retention layer 94 can include a high-density, hydrogen-bonded, fluff/superabsorbent polymer material such as those available as NOVATHIN absorbent cores from EAM Corporation of Jesup, Georgia. These materials include a mixture of fluff pulp and superabsorbent that is formed between two layers of tissue or other nonwoven and densified to form a high density composite between the tissue wraps. Particularly suitable superabsorbent polymers are HYSORB 8760 superabsorbent (BASF of Charlotte, N.C.) and FAVOR SXM 9500 absorbent (available from EVONIK Stockhausen of Greensboro, North Carolina). The composition generally includes no chemical binders. The composition can further include synthetic bonding fibers.

Basis weights can range from 80 to 800 gsm. Density can range from 0.1 to 0.45 g/cc. Particulate content can range from 0-70%. The composition can be embossed with different patterns including smooth, circular, or custom bonding patterns as a part of the densification process.

The hydrogen-bonding process eliminates the use of synthetic fibers and/or latex in combination with ovens to stabilize the web. Instead it relies on the combination of temperature and pressure at the calendering step to initiate hydrogen bonding and thus stabilize the web. The main advantage of this technology is the simplicity of the manufacturing process due to the elimination of expensive unit operations. Other advantages include better containment of particulates such as superabsorbent material and higher efficiency of absorbency due to the absence of materials that affect absorbency such as synthetic fibers and binding agents.

In still another aspect of the present disclosure, one or more of the layers can include a spunlace material. Spunlace materials include the use of meltblown fibers as part of the structure (e.g., laminate). The material is subjected to hydraulic entangling that facilitates entanglement of the various fibers and/or filaments. This results in a higher degree of entanglement and allows the use of a wider variety of other fibrous material in the laminate. Moreover, the use of meltblown fibers can decrease the amount of energy needed to hydraulically entangle the laminate. In spunlace or hydraulic entangling bonding technology, typically a sufficient number of fibers with loose ends (e.g., staple fibers and wood fibers), small diameters, and high fiber mobility are incorporated in the fibrous webs to wrap and entangle around fiber filament, foam, net, etc., cross-over points. Without such fibers, bonding of the web can be poor. Continuous large diameter filaments that have no loose ends and are less mobile have normally been considered poor fibers for entangling. Meltblown fibers, however, have been found to be effective for wrapping and entangling or intertwining. This is due to the fibers having small diameters and a high surface area, and the fact that when a high enough energy flux is delivered from the jets, fibers break up, are mobilized, and entangle other fibers. This phenomenon occurs regardless of whether meltblown fibers are in the aforementioned layered forms or in admixture forms.

The use of meltblown fibers (e.g., microfibers) provides an improved product in that the intertwining among the meltblown fibers and other, e.g., fibrous, material in the laminate is improved. Thus, due to the relatively great length and relatively small thickness of the meltblown fibers, entangling of the meltblown fibers around the other material in the laminate is enhanced. Moreover, the meltblown fibers have a relatively high surface area, small diameters, and are sufficient distances apart from one another to allow other fibrous material in the laminate to freely move and wrap around and within the meltblown fibers. In addition, because the meltblown fibers are numerous and have a relatively high surface area, small diameter, and are nearly continuous, such fibers are excellent for bonding loose fibers (e.g., wood fibers and staple fibers) to them. Anchoring or laminating such fibers to meltblown fibers requires relatively low amounts of energy to entangle.

The use of hydraulic entangling techniques to mechanically entangle (e.g., mechanically bond) the fibrous material, rather than using only other bonding techniques, including other mechanical entangling techniques, provides a composite nonwoven fibrous web material having increased strength, integrity, and hand and drape, and allows for better control of other product attributes, such as absorbency, wet strength, etc.

One example of a spunlace fabric is OPTIMAL GSM 30-250 100% Rayon fabric available from Baiksan Lintex Co., Ltd. of Siheung-City, South Korea.

Spunlace fabric generally refers to a material that has been subjected to hydraulic entangling. Although spunlace fabric is relatively inexpensive, breathable, and can be deformed, the deformation is generally considered to be permanent and can be described as non-recoverable stretch. Nonwoven webs of very small diameter fibers or microfibers have long been known to be permeable to air and water vapor while remaining relatively impermeable to liquids and/or particulates. Useful webs of small diameter fibers can be made by extruding non-elastomeric thermoplastic polymers utilizing fiber forming processes such as, for example, meltblowing processes. Although nonwoven webs of meltblown fibers formed from non-elastomeric polymers are relatively inexpensive and breathable, those highly entangled webs tend to respond poorly to stretching forces. Elongation that occurs in such materials is generally considered to be a permanent, non-recoverable elongation (i.e., non-recoverable stretch). For example, nonwoven webs made from conventional thermoplastic polypropylene are usually considered to have non-recoverable stretch.

In yet another aspect of the present disclosure, one or more layers of the absorbent composite 84 can include a foam material such as those obtainable from The Dow Chemical Company of Midland, Mich. Representative absorbent foam materials are described in U.S. Pat. Nos. 6,627,670 B2 to Mork et al., 6,071,580 to B1 and et al., 7,439,276 B2 to Strandburg et al., and in PCT Publication Nos. WO2008/036942A2 to Vansumeren et al., WO2007/011728A2 to Kim et al., WO2008/052122A1 to Menning, and WO2008/100842A1 to Stockton et al., which are incorporated herein in their entirety by reference thereto to the extent they do not conflict herewith.

Such absorbent polymeric foam materials have a hydrophilic, flexible, polymeric foam structure of interconnected open-cells. A feature that can be useful in defining preferred polymeric foams is the cell structure. Foam cells, and especially cells that are formed by polymerizing a monomer-containing oil phase that surrounds relatively monomer-free water-phase droplets, will frequently be substantially spherical in shape. These spherical cells are connected to each other by openings, which are referred to hereafter as holes between cells. Both the size or “diameter” of such spherical cells and the diameter of the openings (holes) between the cells are commonly used for characterizing foams in general. Because the cells and holes between the cells in a given sample of polymeric foam will not necessarily be of approximately the same size, average cell and hole sizes (i.e., average cell and hole diameters) will often be specified.

Cell and hole sizes are parameters that can impact a number of important mechanical and performance features of the foams, including the fluid wicking properties of these foams, as well as the capillary pressure that is developed within the foam structure. A number of techniques are available for determining the average cell and hole sizes of foams. The most useful technique involves a simple measurement based on the scanning electron photomicrograph of a foam sample. The foams, useful as absorbents for aqueous fluids, will preferably have an average cell size of from about 20 to about 200 μm, more preferably from about 30 to about 190 μm, and most preferably from about 80 to about 180 μm; and a number average hole size of from about 5 to about 45 μm, preferably from about 8 to about 40 μm, and most preferably from about 20 to about 35 μm.

For example, U.S. Pat. No. 6,071,580 to Bland et al. describes an absorbent, extruded, open cell thermoplastic foam. The foam has an open cell content of about 50 percent or more and an average cell size of up to about 1.5 millimeters. The foam is capable of absorbing a liquid at about 50 percent or more of its theoretical volume capacity when absorbing a liquid. The foam preferably has an average equivalent pore size of about 5 micrometers or more. The foam preferably has a structure substantially of cell walls and cell struts. Also described is a method for absorbing a liquid employing the foam by elongation of the extrudate of the extrusion die, and a method of enhancing absorbency of an open cell foam by applying a surfactant to an exposed surface of the foam such that the surfactant remains at the surface and does not infiltrate a substantial distance into the foam.

Suitable foam materials can also include various types of foams, including, but not limited to, thermoplastic foams, high internal phase emulsion (HIPE) foams and inverse high internal phase emulsion (I-HIPE) foams, and other suitable polymeric foams, including, but not limited to, those disclosed in U.S. Pat. Nos. 7,053,131 to Ko et al., 7,358,282 to Krueger et al., and 5,692,939 to DesMarais et. al., and in U.S. Patent Application Publication No. US2006/0148917 to Radwanski et al., which are incorporated herein in their entirety by reference thereto to the extent they do not conflict herewith. One such example of a suitable foam material is a polyurethane foam with a negative Poisson ratio. Materials typically used as backsheet materials in conventional feminine pads can also be suitable. Examples of extensible backsheet materials are described in U.S. Pat. No. 5,611,790 to Osborn, Ill. et al., which is incorporated herein in its entirety by reference thereto to the extent it does not conflict herewith. Further examples of suitable absorbent foam materials are described in U.S. Patent Application Publication No. US2006/0246272 to Zhang et al., which is incorporated herein in its entirety by reference thereto to the extent it does not conflict herewith.

In another aspect of the present disclosure, the retention layer 94 can include a superabsorbent polymer/adhesive composite material, including a stretch superabsorbent polymer/adhesive composite material. Such composites are described in U.S. Pat. Nos. 5,411,497 to Tanzer et al., 5,433,715 to Tanzer et al., and 7,247,215 to Schewe et al., and U.S. Patent Application Publication No. 2005/0096623A1 to Nhan et al., which are incorporated herein in their entirety by reference thereto to the extent they do not conflict herewith.

In still another aspect of the present disclosure, one or more of the layers of the absorbent composite 84 can include a bonded-carded web (BCW) such as those described in U.S. Pat. Nos. 5,364,382 to Latimer et al., 5,429,629 to Latimer et al., and 5,486,166 to Bishop et al., which are incorporated herein in their entirety by reference thereto to the extent they do not conflict herewith. Typical basis weights for BCW materials include those in the range 30-300 gsm. These patents describe BCW surge technology and how BCW surge materials are made.

In another aspect of the present disclosure, one or more of the layers of the absorbent composite 84 can include meltblown microfiber material. An example of such a meltblown microfiber material is the 50 gsm Meltblown Strip white hydrophilic meltblown available from the Yuhan-Kimberly Kimcheon Nonwoven Mill of KimCheon City, KyungSangBuk-Do, Korea. This material can have a polypropylene fiber diameter of 1-5 microns, a composite density of 0.124-0.218 g/cc, a pore size of 15-18 microns (21-30 microns maximum), and can further include a wettable surfactant such as AEROSOL GPG surfactant available from Cytec Industries Inc. of West Paterson, New Jersey.

The development of highly absorbent articles for blood and blood-based fluids such as catamenial pads (e.g., sanitary napkins), tampons, wound dressings, bandages and surgical drapes can be challenging. Compared to water and urine, blood and blood based fluids such as menses are relatively complex mixtures of dissolved and undissolved components (e.g., erythrocytes or red blood cells). In particular, blood-based fluids such as menses are much more viscous than water and urine. This higher viscosity hampers the ability of conventional absorbent materials to efficiently and rapidly transport these blood-based fluids to regions remote from the point of initial discharge. Undissolved elements in these blood-based fluids can also potentially clog the capillaries of these absorbent materials. This makes the design of appropriate absorbent systems for blood-based fluids such as menses particularly difficult.

In the case of catamenial pads, women have come to expect a high level of performance in terms of comfort and fit, retention of fluid, and minimal staining. Above all, leakage of fluid from the pad onto undergarments is regarded as unacceptable. Improving the performance of such catamenial pads continues to be a formidable undertaking, although a number of improvements have been made in both catamenial structures, and materials used in such structures. However, eliminating leakage, particularly along the inside of the thighs, without compromising fit and comfort, has not always met the desired needs of the consumer.

The absorbent structures of current catamenial (e.g., sanitary napkin) pads have typically comprised one or more fibrous layers for acquiring the discharged menstrual fluid from the permeable topsheet and distributing it to an underlying storage area. Absorbent structures for relatively thin versions of prior catamenial products usually comprise a fluid acquisition or intake layer that is adjacent to the permeable topsheet. This intake layer typically is made from an air-laid-tissue web or a synthetic nonwoven. Underlying this intake layer is the main absorbent core that is typically made from air-laid or wet-laid tissue.

Prior catamenial absorbent structures made from fibrous layers have a number of problems. One is the difficulty in ensuring adequate topsheet dryness. Such structures have also had a greater chance of causing panty and body soiling. This is because the absorbent structure lacks resilience, leading to bunching of the pad. This lack of resilience, and consequent bunching, has also caused these prior catamenial pads to provide poorer fit and comfort for the user. The issue that conventional catamenial absorbent structures and conventional absorbent fibrous webs have not solved this problem was recognized in U.S. Pat. No. 5,849,805 to Dyer.

One attempted solution replaced fibrous intake and retention layers with foam, such as the INFINICEL foam used in ALWAYS INFINITY Regular pads available from The Procter and Gamble Company of Cincinnati, Ohio. Such foams tend to be more expensive than fibrous webs.

Coform nonwoven webs, which are composites of a matrix of meltblown fibers and an absorbent material (e.g., fluff pulp fibers), have been used as an absorbent layer in a wide variety of applications, including absorbent articles, absorbent dry wipes, wet wipes, and mops. Most conventional coform webs employ meltblown fibers formed from polypropylene homopolymers. One problem sometimes experienced with such coform materials, however, is that coform materials might not be sufficiently resilient when subjected to bending forces. For example, when a coform wiper is crumpled, the coform material might not return to its original flat, unwrinkled state, as shown in FIGS. 4 and 5. As another example, a coform material used as an absorbent core in personal care absorbent product can have a tendency for bunching.

As such, the improved coform nonwoven web disclosed herein can be used in a variety of applications, and shows improved resistance to bending forces and demonstrates a tendency to return to a flat state after being folded. Such an improved coform nonwoven web can be combined with various other materials to produce a next-generation absorbent composite for use in personal care absorbent articles as shown in FIGS. 6 and 7.

The present inventors undertook intensive research and development efforts with respect to improving absorbent articles and have developed absorbent composites for use in an absorbent core that has adequate wet and dry resilience and adequate absorbency, without the primary use of expensive foams. The present inventors also found that they can tailor these properties through combining resilient coform with other materials to deliver enhanced resiliency and absorbency properties.

Products incorporating the materials described herein yielded unexpected and surprising results when tested with consumers against a commercial product that replaced a fibrous web with foam. Menstrual pads including a resilient coform of 215 gsm 50% VISTAMAXX polymer/50% fluff pulp blend intake layer 86 paired with a retention layer 94 including EAM 150 gsm NOVATHIN high-density, hydrogen-bonded, fluff/superabsorbent polymer material, including 25% superabsorbent, were compared to commercially-available ALWAYS INFINITY Regular pads incorporating INFINICEL HIPE foam. Despite these two different technology approaches and costs, both products received the equivalent overall purchase intent along with the same perception of comfort and absorbency. This result was unexpected because the more expensive INFINICEL foam was hypothesized to deliver benefits above and beyond the lower cost resilient coform/NOVATHIN material combination tested. Other commercially-available products that did not include a resilient coform layer or INFINICEL foam were tested in the same manner, but did not deliver the same comfort as the two products described in this paragraph.

As described above, prior catamenial absorbent structures employing fibrous webs have had a greater chance of causing panty and body soiling because the absorbent structure lacks resilience, leading to bunching of the pad. This lack of resilience, and consequent bunching, has also caused these prior catamenial pads to provide poorer fit and comfort for the user. On the contrary, the absorbent structure disclosed herein solves such issues, as illustrated in Table 1.

TABLE 1
Results of Consumer Testing

| | | | |
|---|---|---|---|
| Intake Layer: | 108 gsm VISTAMAXX | ALWAYS INFINITY | Conventional Pad: |
| | 2330 polymer, 108 gsm | HIPE Foam Pad | ALWAYS Ultrathin |
| | CF 405 pulp fiber | | Regular |
| Retention Layer: | EAM NOVATHIN | INFINICEL Foam | |
| | J1501825DTNB material | | |
| | with 25% superabsorbent | | |
| Overall Comfort | 4.3 | 4.2 | 3.8 |
| Overall | 4.2 | 4.2 | 4.0 |
| Absorbency | | | |

These numbers above represent a monadic rating on a five point scale, with 5 being better. The results in the first two columns have no statistically-significant differences. The results in the third column show a statistically-significant difference from the first two columns, and demonstrate worse results.

The absorbent composite 84 of the present disclosure can be better understood with reference to the following absorbent composite test methods and examples.

### Absorbent Composite Test Method: Side Compression Test

The side compression test is used to measure the flexibility and resiliency of the feminine pad sample by compressing and then de-compressing the pad sample sidewise. To perform this test, a CRE (Constant Rate of Elongation) tensile tester (such as MTS SINTECH 500/S model, Serial No. 500S/062696/203 or equivalent) is used. The data acquisition software is MTS TESTWORKS for Windows Ver. 4.11 C (MTS Systems Corporation, Eden Prairie, Minn.). The load cell is selected from either a 50 Newton or 100 Newton maximum, depending on the peak force value of the sample being tested, such that the majority of peak load values fall between 10-90% of the load cell's full scale value. In this test, both edges of the pad (i.e. the liner and outer cover laminate) are clamped between top and bottom grips of the tensile tester with the center of the sample aligned with the center of the grips and the sample centered between the grips. The grip face width is 3 inches (76.2 mm), and the approximate height of the grip is 1.0 inch (25.4 mm). The test speed is 5±0.04 inches/min (127±1 mm/min) in both compression and de-compression modes. The initial gauge length is set at 55 mm. When the test starts, the grips move toward each other to compress the sample until the grip distance is 20±1 mm. The grips then return to their initial positions at the conclusion of the test. The sample can be tested dry. Furthermore, the tested dry sample can be wetted with 5 mL of fluid and re-tested for the wet condition.

Pressure vs. distance is plotted to produce a compression curve. Pressure vs. distance is also plotted as the sample is released from compression, producing a decompression curve.

Three test parameters of interest from this tester are as follows. Peak Compression Force (gf) is the maximum force detected in the compression curve up to a compression distance. A higher value indicates a higher force needed to compress the product to a specific thickness. In practical application, a consumer wears a product, compressing it between her legs. A higher peak force suggests that a greater effort is required to compress the product. Compression Energy (gf cm) is the area under the compression curve. A higher value indicates that a product is more difficult to compress. In practical application, this means that more energy is required to compress the product between the legs. This parameter considers all points, not just the peak force. Finally, compression Resiliency (%) is the ratio of decompression to compression area. A higher value indicates greater recovery. In practical application, a consumer wears product, compressing it between her legs. As she releases it, the product returns to original state. This is advantageous for reducing bunching and twisting issues. Ideally with respect to comfort, one wants a product that is easy to compress (low peak force, low energy) and resilient.

The same test method is used to develop the wet compression and resilience data in Table 2 except that 5 ml of menses simulant is added to the dry pad with a syringe spreading the fluid over the full area of the bodyside liner. The menses simulant used is made of swine blood diluted to a hematocrit level of 30% by volume, with sheared, thick egg white added to mimic the mucin component of menses. This simulant is available from Cocalico Biologicals, Inc. of Reamstown, Pa., and is also described in U.S. Pat. Nos. 5,883,231 to Achter et al. and 7,632,258 to Misek et al., which are incorporated herein in their entirety by reference thereto to the extent they do not conflict herewith.

### ABSORBENT COMPOSITE EXAMPLES

### Example A

### Commercially-Available ALWAYS INFINITY Regular Flow Pad

### Example B

### U by KOTEX CLEANWEAR Regular Pad

For the following examples, VISTAMAXX 2330 polymer is available from ExxonMobil Chemical Corp., CF 405 pulp fiber is available from Weyerhaeuser Co., and FAVOR SXM9500 superabsorbent is available from Evonik Stockhausen, Inc.

### Example 1

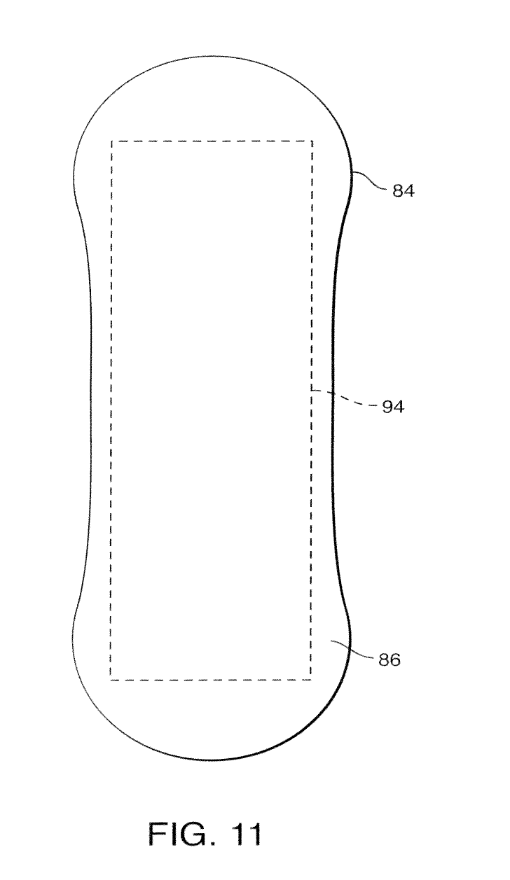

A pad with the shape illustrated in FIG. 11 was manufactured and tested; the pad included a first intake layer (86) including a resilient coform having 108 gsm VISTAMAXX 2330 polymer with 108 gsm CF 405 pulp fiber, and a retention layer (94) including EAM NOVATHIN J1501825DTNB material (a high-density, hydrogen-bonded, fluff/superabsorbent polymer material).

### Example 2

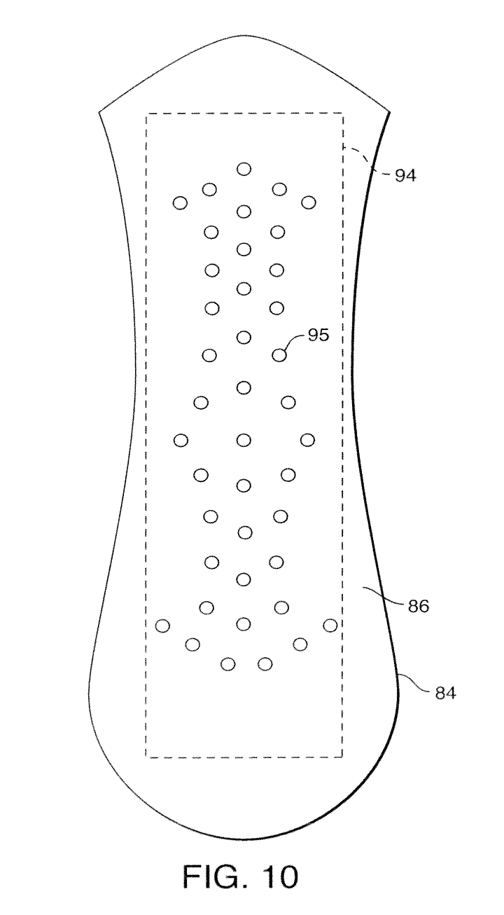

A pad with the shape illustrated in FIG. 10 but without holes (95) was manufactured and tested; the pad included a first intake layer (86) including a resilient coform having 108 gsm VISTAMAXX 2330 polymer with 108 gsm CF 405 pulp fiber, and a retention layer (94) including EAM NOVATHIN J1501825DTNB material (a high-density, hydrogen-bonded, fluff/superabsorbent polymer material).

### Example 3

A pad with the shape illustrated in FIG. 10 but without holes (95) was manufactured and tested; the pad included a first intake layer (86) including a resilient coform having 108 gsm VISTAMAXX 2330 polymer with 108 gsm CF 405 pulp fiber, a retention layer (94) including EAM NOVATHIN J1501825DTNB material (a high-density, hydrogen-bonded, fluff/superabsorbent polymer material), and a distribution layer (96) including 2 layers of 50 gsm meltblown microfiber.

### Example 4

A pad with the shape illustrated in FIG. 10 but without holes (95) was manufactured and tested; the pad included a layer including a resilient coform having 108 gsm VISTAMAXX 2330 polymer with 108 gsm CF 405 pulp fibers, and a second layer including Glatfelter Airlaid DT200.102.

### Example 5

A pad with the shape illustrated in FIG. 10 was manufactured and tested; the pad included a first intake layer (86) including 150 gsm polyolefin foam with a density of 0.07 g/cc and a 0.5 osy spunbond substrate, and a retention layer (94) including 215 gsm coform made of 108 gsm VISTAMAXX 2330 polymer, 75 gsm CF 405 pulp fiber, and 32 gsm FAVOR SXM9500 superabsorbent particles. A hole pattern of 41 holes (95), each 3 mm in diameter, and arranged in the pattern illustrated in FIG. 10 was formed through both layers. The outline of the pad illustrated in FIG. 10 is for context to show the general relationship of the holes (95).

### Example 6

A pad with the shape illustrated in FIG. 10 but without holes (95) was manufactured and tested; the pad included a first intake layer (86) including a resilient coform having 108 gsm VISTAMAXX 2330 polymer with 108 gsm CF 405 pulp fiber, and a retention layer (94) including 215 gsm coform made of 108 gsm VISTAMAXX 2330 polymer, 75 gsm CF 405 pulp fiber, and 32 gsm FAVOR SXM9500 superabsorbent particles.

### Example 7

A pad with the shape illustrated in FIG. 10 but without holes (95) was manufactured and tested; the pad included a first intake layer (86) including a resilient coform having 108 gsm VISTAMAXX 2330 polymer with 108 gsm CF 405 pulp fiber, and a retention layer (94) including a 100 gsm spunlace material.

TABLE 2
Test Results: Side Compression Test

| | | | | Peak Compression | | Compression | | Compression | |
|---|---|---|---|---|---|---|---|---|---|
| | | | | Force (gf) | | Energy (gf*cm) | | Resilience (%) | |
| | | | | | 5 mL | | 5 mL | | 5 mL |
| Ex | Intake Layer | Retention Layer | Distribution Layer | Dry | simulant | Dry | simulant | Dry | simulant |
| A | ALWAYS INFINITY Regular Pad (date code | | | 160 | 106 | 262 | 252 | 44 | 44 |
| | 83394786671506) | | | | | | | | |
| B | U by KOTEX CLEANWEAR Regular Pad (date code | | | 321 | 238 | 685 | 532 | 32 | 29 |
| | BJ925405X1337) | | | | | | | | |
| 1 | resilient coform 108 | EAM NOVATHIN | N/A | 100 | 102 | 285 | 245 | 66 | 52 |
| | gsm VM2330, 108 | J1501825DTNB | | | | | | | |
| | gsm CF 405, no holes | material | | | | | | | |
| 2 | resilient coform 108 | EAM NOVATHIN | N/A | 55 | 49 | 138 | 96 | 47 | 20 |
| | gsm VM2330, 108 | J1501825DTNB | | | | | | | |
| | gsm CF 405, no holes | material | | | | | | | |
| 3 | resilient coform 108 | EAM NOVATHIN | 2 layers of 50 | 103 | 95 | 296 | 264 | 37 | 23 |
| | gsm VM2330, 108 | J1501825DTNB | gsm meltblown | | | | | | |
| | gsm CF 405, no holes | material | microfiber | | | | | | |
| 4 | resilient coform 108 | Glatfelter Airlaid | N/A | 114 | 82 | 336 | 242 | 44 | 33 |
| | gsm VM2330, 108 | DT200.102 | | | | | | | |
| | gsm CF 405, no holes | | | | | | | | |
| 5 | 150 gsm polyolefin | Coform: 108 | N/A | 104 | 99 | 231 | 200 | 35 | 40 |
| | foam | gsm VM2330, | | | | | | | |
| | density = 0.07 g/ccand | 75 gsm CF 405, | | | | | | | |
| | a 0.5 osy spunbond | 32 gsm | | | | | | | |
| | substrate, with hole | SXM9500, with | | | | | | | |
| | pattern | hole pattern | | | | | | | |
| 6 | resilient coform 108 | coform: 108 gsm | N/A | 65 | 60 | 159 | 149 | 26 | 23 |
| | gsm VM2330, 108 | VM2330, 75 gsm | | | | | | | |
| | gsm CF 405, no holes | CF 405, 32 gsm | | | | | | | |
| | | SXM9500 | | | | | | | |
| 7 | resilient coform 108 | 100 gsm | N/A | 62 | 60 | 140 | 144 | 22 | 13 |
| | gsm VM2330, 108 | spunlace from | | | | | | | |
| | gsm CF 405, no holes | Baiksan Lintex | | | | | | | |
| | | Co., Ltd. | | | | | | | |

VM2330 refers to VISTAMAXX 2330 polymer available from ExxonMobil Chemical Corp.
CF 405 refers to CF 405 pulp fiber available from Weyerhaeuser Co.
SXM9500 refers to FAVOR SXM9500 superabsorbent available from Evonik Stockhausen, Inc.
Because pad shape can also impact the measured compressibility and resilience, the pad shape of FIG. 11 was used for Example 1, and the pad shape of FIG. 10 was used for Examples 2-7.

Each of the above examples including resilient coform had peak forces comparable to or lower than that of ALWAYS INFINITY Regular Pad.

While the disclosure has been described in detail with respect to the specific aspects thereof, it will be appreciated that those skilled in the art, upon attaining an understanding of the foregoing, can readily conceive of alterations to, variations of, and equivalents to these aspects. Accordingly, the scope of the present disclosure should be assessed as that of the appended claims and any equivalents thereto. In addition, it should be noted that any given range presented herein is intended to include any and all lesser included ranges. For example, a range of from 45-90 would also include 50-90, 45-80, 46-89, and the like.

1. An absorbent composite disposed in an absorbent article between a topsheet and a backsheet, the absorbent composite comprising:
a first intake layer disposed between the topsheet and the backsheet; and
a retention layer disposed between the topsheet and the backsheet, wherein one of the first intake layer and the retention layer includes a resilient coform material.

2. The absorbent composite of claim 1, wherein the first intake layer includes a resilient coform material, and wherein the retention layer includes one of a coform material, a resilient coform material, a bonded-carded web (BCW) material, and an airlaid material.

3. The absorbent composite of claim 2, wherein the retention layer further includes superabsorbent material.

4. The absorbent composite of claim 2, wherein the retention layer further includes fluff pulp.

5. The absorbent composite of claim 1, wherein the first intake layer includes a resilient coform material, and wherein the retention layer includes one of a high-density, hydrogen-bonded, fluff/superabsorbent polymer material, a spunlace material, a superabsorbent polymer/adhesive composite material, and a foam material.

6. The absorbent composite of claim 5, wherein the retention layer further includes fluff pulp.

7. The absorbent composite of claim 1, wherein the retention layer includes a resilient coform material, and wherein the first intake layer includes one of a coform material, a resilient coform material, an airlaid material, a bonded-carded web (BCW) material, and a foam material.

8. The absorbent composite of claim 7, wherein the first intake layer further includes fluff pulp.

9. The absorbent composite of claim 7, wherein the resilient coform material in the retention layer includes superabsorbent material.

10. The absorbent composite of claim 1, further comprising a distribution layer disposed between the topsheet and the backsheet, the distribution layer including one of a meltblown microfiber material, a spunlace material, and a foam material.

11. The absorbent composite of claim 1, wherein the first intake layer includes a resilient coform material, and wherein the retention layer includes a high-density, hydrogen-bonded, fluff/superabsorbent polymer material.

12. The absorbent composite of claim 1, wherein the first intake layer includes a resilient coform material, and wherein the retention layer includes an airlaid material.

13. The absorbent composite of claim 1, wherein both the first intake layer and the retention layer include a resilient coform material.

14. The absorbent composite of claim 13, wherein the retention layer further includes superabsorbent material.

15. The absorbent composite of claim 1, further comprising a second intake layer disposed generally in parallel with the first intake layer, the second intake layer including one of an airlaid material, a bonded-carded web (BCW) material, a resilient coform material, and a foam material.

16. The absorbent composite of claim 1, wherein the intake layer is between the topsheet and the retention layer, and wherein the retention layer is between the backsheet and the intake layer.

17. An absorbent composite disposed in an absorbent article between a topsheet and a backsheet, the absorbent composite comprising:
a first intake layer including one of a coform material, a resilient coform material, an airlaid material, a bonded-carded web (BCW) material, and a foam material; and
a retention layer disposed between the topsheet and the backsheet, the retention layer including one of a coform material, a resilient coform material, an airlaid material, a high-density, hydrogen-bonded, fluff/superabsorbent polymer material, a spunlace material, a superabsorbent polymer/adhesive composite material, and a foam material, wherein one of the first intake layer and the retention layer includes a resilient coform material.

18. An absorbent composite adapted to be disposed in an absorbent article between a topsheet and a backsheet, the absorbent composite comprising:
a first intake layer including a resilient coform material; and
a retention layer disposed between the topsheet and the backsheet, the retention layer including one of a coform material, a resilient coform material, an airlaid material, a high-density, hydrogen-bonded, fluff/superabsorbent polymer material, a spunlace material, a superabsorbent polymer/adhesive composite material, and a foam material.

19. An absorbent personal care article having a topsheet and a backsheet, the article comprising:
an absorbent composite disposed between the topsheet and the backsheet, the absorbent composite including a first intake layer including a resilient coform material, and a retention layer disposed between the topsheet and the backsheet, the retention layer including one of a coform material, a resilient coform material, an airlaid material, a high-density, hydrogen-bonded, fluff/superabsorbent polymer material, a spunlace material, a superabsorbent polymer/adhesive composite material, and a foam material.

20. A method for making absorbent personal care article having an absorbent composite, the method comprising:
merging a stream of an absorbent material with a stream of meltblown fibers to form a composite stream;
collecting the composite stream on a forming surface to form a resilient coform nonwoven web; and
combining the resilient coform nonwoven web with a topsheet and a backsheet.

21. An absorbent composite adapted for use in an absorbent article having a topsheet and a backsheet, the absorbent composite comprising:
an intake layer including a foam material disposed between the topsheet and the backsheet, the intake layer having a plurality of holes therethrough; and
a retention layer disposed between the topsheet and the backsheet, wherein the retention layer includes a resilient coform material.

22. The absorbent composite of claim 21, wherein the retention layer has a plurality of holes therethrough.
